SEQUENCE LISTING (3 sequences)

SEQ ID NO: 1 - protein, 755 aa, Artificial Sequence
  Description of Artificial Sequence Synthetic polypeptide
MSPILGYWKIKGLVQPTRLLLEYLEEKYEEHLYERDEGDKWRNKKFELGLEFPNLPYYIDGDVKLTQSMAIIRYIADKHNMLGGSPKERAEISMLEGAVLDIRYGVSRIAYSKDFETLKVDFLSKLPEMLKMFEDRLSHKTYLNGDHVTHPDFMLYDALDVVLYMDPMSLDAFPKLVSFKKRIEAIPQIDKYLKSSKYIAWPLQGWQATFGGGDHPPKSDLVPRGSPEFPGRLERPHRDGGGSENLYFQGSGGASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLQLGTQPELIQDAGGGVRMSGAGTASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLASHMASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLQLGTQPELIQDAGGGVRMSGAGTASRGVNKVILVGNLGQDPEVRYMPNGGAVANITLATSESWRDKATGEMKEQTEWHRVVLFGKLAEVASEYLRKGSQVYIEGQLRTRKWTDQSGQDRYTTEVVVNVGGTMQMLGGSC

SEQ ID NO: 2 - protein, 400 aa, Artificial Sequence
  Description of Artificial Sequence Synthetic polypeptide
MGSSHHHHHHSSGLVPRGSHMSPSPVPGSQNVPAPAVKKISQYAAQRRTTLNNYNQLFTDALDILAENDELRENEGSALAFMRASSVLKSLPFPITSMKDTEGIPNLGDKVKSIIEGIIEDGESSEAKAVLNDERYKSFKLFTSVFGVGLKTAEKWFRMGFRTLSKIQSDKSLRFTQMQKAGFLYYEDLVSAVNRPEAEAVSMLVKEAVVTFLPDALVTMTGGFRRGKMTGHDVDFLITSPEATEDEEQQLLHKVTDFWKQQGLLLYGDILESTFEKFKQPSRKVDALDHFQKAFLILKLDHGRVHSEKSGQQEGKGWKAIRVDLVMAPYDRRAFALLGWTGSRQFERDLRRYATHERKMMLDNHALYDRTKRVFLEAESEEEIFAHLGLDYIEPWERNA

SEQ ID NO: 3 - DNA, 18 bp, Artificial Sequence
  Description of Artificial Sequence Synthetic primer
tttttttttttttttttt

(19) United States
(12) Patent Application Publication
(10) Pub. No.: US 2019/0360013 A1
(43) Pub. Date: Nov. 28, 2019

(54) Processive Template Independent DNA Polymerase Variants

(71) Applicant: PRESIDENT AND FELLOWS OF HARVARD COLLEGE, Cambridge, MA (US)

(72) Inventors: Kettner John Frederick GRISWOLD, JR., Brookline, MA (US); Brian M. TURCZYK, Westford, MA (US); Daniel Jordan WIEGAND, Somerville, MA (US); George M. CHURCH, Brookline, MA (US); Alexander GARRUSS, Cambridge, MA (US)

(73) Assignee: President and Fellows of Harvard College, Cambridge, MA (US)

(21) Appl. No.: 16/465,816

(22) PCT Filed: Dec. 4, 2017
(86) PCT No.: PCT/US17/64517

§ 371 Date: May 31, 2019

Related U.S. Application Data
(60) Provisional application No. 62/429,344, filed on Dec. 2, 2016.

(51) Int. Cl. C12P 19/34; C12N 9/12

(52) U.S. Cl. CPC C12P 19/34; C12Y 207/07031; C12N 9/1264

(57) ABSTRACT

An enzymatic method of making a polynucleotide is provided. The method includes combining a selected nucleotide triphosphate, one or more cations, a template-independent polymerase, and an associated processivity factor in an aqueous reaction medium including a target substrate comprising an initiator sequence and having a 3′ terminal nucleotide attached to a single stranded portion, such that the template-independent polymerase and the associated processivity factor interact with the target substrate under conditions which covalently add one or more of the selected nucleotide triphosphate to the 3′ terminal nucleotide. Also provided are mutant template-independent polymerases having a processivity factor attached thereto.

### RELATED APPLICATION DATA

This application claims priority to U.S. Provisional Application No. 62/429,344 filed on Dec. 2, 2016, which is hereby incorporated herein by reference in its entirety for all purposes.

### STATEMENT OF GOVERNMENT INTERESTS

This invention was made with government support under grant numbers RM1HG008525 and R01MH103910 awarded by National Institutes of Health. The government has certain rights in the invention.

### FIELD

The present invention relates in general to methods of making polynucleotides using template independent DNA polymerase variants.

### BACKGROUND

Template-independent polymerases have increasingly been used in polynucleotide synthesis methods. Yet, a need exists for methods of making polynucleotides using template independent DNA polymerase variants with improved functionality.

### SUMMARY

According to one aspect, the present disclosure provides an enzymatic method of making a polynucleotide. In one embodiment, the method includes combining a selected nucleotide triphosphate, one or more cations, a template-independent polymerase, and an associated processivity factor in an aqueous reaction medium including a target substrate comprising an initiator sequence and having a 3′ terminal nucleotide attached to a single stranded portion, such that the template-independent polymerase and the associated processivity factor interact with the target substrate under conditions which covalently add one or more of the selected nucleotide triphosphate to the 3′ terminal nucleotide. In another embodiment, the method further includes repeatedly introducing a subsequent selected nucleotide triphosphate to the aqueous reaction medium under conditions which enzymatically add one or more of the subsequent selected nucleotide triphosphate to the target substrate until the polynucleotide is formed. In one embodiment, the processivity factor increases processivity of the template-independent polymerase. In certain embodiments, the processivity factor comprises one or more binding units. In one embodiment, the processivity factor binds to and translocates across the target substrate. In another embodiment, the processivity factor binds to and reptates across the target substrate. In one embodiment, the processivity factor and the template-independent polymerase bind to the target substrate. In exemplary embodiments, the processivity factor and the template-independent polymerase bind to the target substrate with an affinity greater than the template-independent polymerase alone or without the processivity factor. In one embodiment, the processivity factor and the template-independent polymerase comprise a fusion protein. In some embodiments, the processivity factor is attached to the template-independent polymerase at a location on the template-independent polymerase which facilitates processing of the target substrate by the template-independent polymerase. In other embodiments, the processivity factor is attached by a covalent or noncovalent bond to the template-independent polymerase at a location on the template-independent polymerase which facilitates processing of the target substrate by the template-independent polymerase. In some embodiments, the processivity factor is attached to the template-independent polymerase through a linker at a location on the template-independent polymerase which facilitates processing of the target substrate by the template-independent polymerase. In other embodiments, the processivity factor includes a polypeptide binding domain that binds to the template-independent polymerase. In some embodiments, the template-independent polymerase includes a polypeptide binding domain that binds to the processivity factor. In other embodiments, the template-independent polymerase and the processivity factor each include one member of a binding pair wherein the template-independent polymerase and the processivity factor are attached via the binding pair. In certain embodiments, the template-independent polymerase and the processivity factor are crosslinked via a crosslinker. In exemplary embodiments, the template-independent polymerase and the processivity factor are crosslinked via sulfhydryl crosslinking. In some embodiments, the template-independent polymerase and the processivity factor are attached via protein conjugation. In other embodiments, the template-independent polymerase and the processivity factor are immobilized relative to one another in an orientation which facilitates processing of the substrate by the template-independent polymerase. In some embodiments, the template-independent polymerase and the processivity factor are immobilized relative to one another on a substrate in an orientation which facilitates processing of the substrate by the template-independent polymerase. In other embodiments, the template-independent polymerase and the processivity factor are co-localized on a substrate in an orientation which facilitates processing of the substrate by the template-independent polymerase. In some embodiments, the template-independent polymerase is a template-independent DNA or RNA polymerase. In one embodiment, the template-independent polymerase is a template-independent DNA polymerase. In another embodiment, the template-independent polymerase is a terminal deoxynucleotidyl transferase (TdT). In some embodiments, the template-independent polymerase is a TdT of the polX family of DNA polymerases. In other embodiments, the TdT is a mammalian TdT or TdT from other non-mammalian species. In some embodiments, the TdT is a member of the archaeo-eukaryotic primase (AEP) superfamily. In other embodiments, the TdT is a PolpTN2 or a C-terminal truncated PolpTN2, a PriS, a nonhomologous end joining archaeo-eukaryotic primase, a mammalian Polθ, or a eukaryotic PrimPol. In some embodiments, the template-independent polymerase is a mutant where one or more cysteine residues are replaced by one or more non-cysteine residues. In other embodiments, the template-independent polymerase is a mutant where one or more or a plurality or all naturally occurring cysteine residues are replaced by one or more non-cysteine residues. In some embodiments, the template-independent polymerase is a mutant where one or more or a plurality or all naturally occurring cysteine residues are replaced by one or more non-cysteine residues and a surface accessible cysteine residue is provided. In other embodiments, the template-independent polymerase is a mutant where one or more non-cysteine residues are replaced by one or more cysteine residues. In some embodiments, the template-independent polymerase is a mutant having one or more surface accessible cysteine residues. In other embodiments, the template-independent polymerase is a mutant having at most one surface accessible cysteine residue. In some embodiments, the template-independent polymerase and the processivity factor each include a mutant surface accessible cysteine residue which connects the template-independent polymerase to the processivity factor. In other embodiments, the processivity factor comprises a prokaryotic or eukaryotic single stranded DNA binding protein. In some embodiments, the processivity factor comprises a prokaryotic single stranded DNA binding protein. In exemplary embodiments, the processivity factor comprises an E. coli single stranded DNA binding protein.

According to another aspect, the present disclosure provides mutant template-independent polymerases each having one or more mutations within the template-independent polymerase. In some embodiments, the mutant template-independent polymerase has one or more mutations from a cysteine residue to a non-cysteine residue. In other embodiments, the mutant template-independent polymerase has one or more mutations from a non-cysteine residue to a cysteine residue. In some embodiments, the mutant template-independent polymerase has one or more mutations from a non-cysteine residue to a surface accessible cysteine residue. In other embodiments, the mutant template-independent polymerase has a mutation from a non-cysteine residue to a surface accessible cysteine residue. In some embodiments, the mutant template-independent polymerase has a mutation from a non-cysteine residue to at most one surface accessible cysteine residue.

According to still another aspect, the present disclosure provides a macromolecule comprising a template-independent polymerase having a processivity factor attached thereto. In certain embodiments, the template-independent polymerase is a template-independent DNA or RNA polymerase. In one embodiment, the template-independent polymerase is a template-independent DNA polymerase. In an exemplary embodiment, wherein the template-independent polymerase is a terminal deoxynucleotidyl transferase (TdT). In some embodiments, the template-independent polymerase is a TdT of the polX family of DNA polymerases. In other embodiments, the TdT is a mammalian TdT. In some embodiments, the TdT is a member of the archaeo-eukaryotic primase (AEP) superfamily. In other embodiments, the TdT is a PolpTN2 or a C-terminal truncated PolpTN2, a PriS, a nonhomologous end joining archaeo-eukaryotic primase, a mammalian Polθ, or a eukaryotic PrimPol. In some embodiments, the template-independent polymerase is a mutant where one or more cysteine residues is replaced by a non-cysteine residue. In other embodiments, the template-independent polymerase is a mutant where one or more non-cysteine residues is replaced by a cysteine residue. In some embodiments, the template-independent polymerase is a mutant having one or more surface accessible cysteine residues. In other embodiments, the template-independent polymerase is a mutant having at most one surface accessible cysteine residue. In some embodiments, the template-independent polymerase is attached to the processivity factor by a mutant surface accessible cysteine residue. In certain embodiments, the processivity factor comprises a prokaryotic or eukaryotic single stranded DNA binding protein. In other embodiments, the processivity factor comprises a prokaryotic single stranded DNA binding protein. In exemplary embodiments, the processivity factor comprises an E. coli single stranded DNA binding protein.

Embodiments of the present disclosure are directed to a system for making a polynucleotide including a selected nucleotide triphosphate, one or more cations, a template-independent polymerase, and an associated processivity factor in an aqueous reaction medium including a target substrate comprising an initiator sequence and having a 3′ terminal nucleotide attached to a single stranded portion.

Further features and advantages of certain embodiments of the present invention will become more fully apparent in the following description of embodiments and drawings thereof, and from the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The foregoing and other features and advantages of the present embodiments will be more fully understood from the following detailed description of illustrative embodiments taken in conjunction with the accompanying drawings in which:

FIG. 1 depicts a schematic representation of an exemplary covalent attachment of a single stranded binding protein (SSB) to a template independent DNA polymerase.

FIGS. 2A & 2B show results of purified TdT and its activity. FIG. 2A shows a molecular weight analysis of His-Tag mTdT purified from crude lysate with Tris-Gly gel electrophoresis under denaturing conditions. Lane 1 indicates the removal of unwanted protein from the crude lysate sample in Lane 2. Approximately 1000 ng of material was loaded on the gel. FIG. 2B shows a TBE-Urea gel electrophoresis indicating initiator extension after 60 minutes from purified His-Tag mTdT in comparison to the commercially available Bovine TdT (New England Biolabs, Inc.) with an equalmolar mixture of dNTPs Approximately 100 ng of material was loaded on the gel.

FIGS. 3A & 3B show results of His-tag mTdT activity. FIG. 3A shows the reaction efficiency for individual nucleotide (dATP, dGTP, dTTP, dCTP at 0.1 mM concentration) incorporation by His-tag mTDT was determined by calculating the total single stranded DNA (ssDNA) concentration from the average Relative Fluorescence Units (RFU) value of a nucleic acid stain over 80 minutes. FIG. 3B shows the fold-increase in ssDNA concentration at 80 minutes for each nucleotide was then calculated using the initial concentration of ssDNA at 0 min for several ratios of extended polynucleotide to the initiator fragment.

FIG. 4 shows results of nucleotide incorporation activity of a mTdT compared to a non-cysteine mTdT variant. The nucleotide incorporation activity of a mTDT 388 variant in which all 7 native cysteine residues were replaced with non-cysteine residues was compared to native mTDT 388's incorporation activity. Reactions consisted of dNTPs at a concentration of 0.5 mM and a 20-nt initiation sequence. The average RFU was measured and plotted as a function of incubation time. Measurements were taken every 1 minute until reaction completion at 30 minutes.

FIG. 5A-E show the results of a kinetic analysis of purified wild-type and R454A human TdT with different divalent cations at increasing concentrations. A poly-dT (18) initiator oligonucleotide was used at 10 pmol and dATP was supplemented into the reaction at 100 uM. In the barplot (A), the overall rates of enzyme activity are plotted for the WT hTdT and the rates for hTdT R454A are plotted in barplot (B). These rates were derived from the RFU for ssDNA production measured in real time over 30 minutes as shown in scatterplots (C) for WT hTdT and (D) for hTdT R454A. In the boxplots (E), the average rate of ssDNA production for all divalent cation concentrations were compared for Mn²⁺, Mg²⁺, and Co²⁺ between the WT hTdT and hTDT454A.

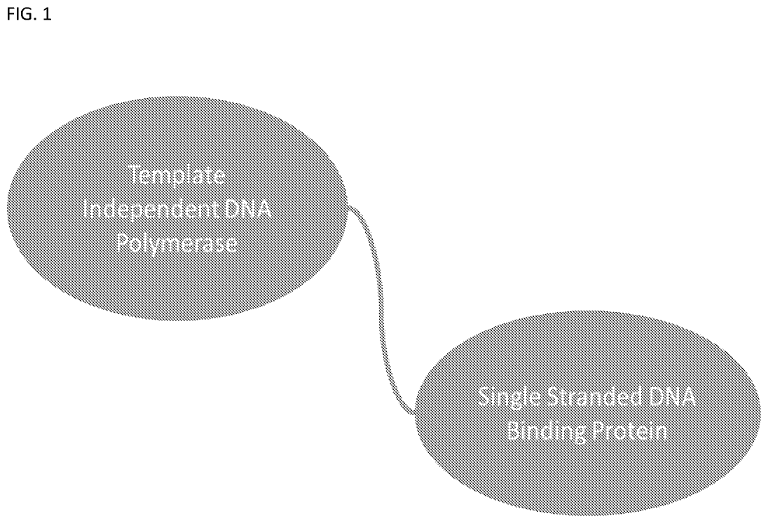

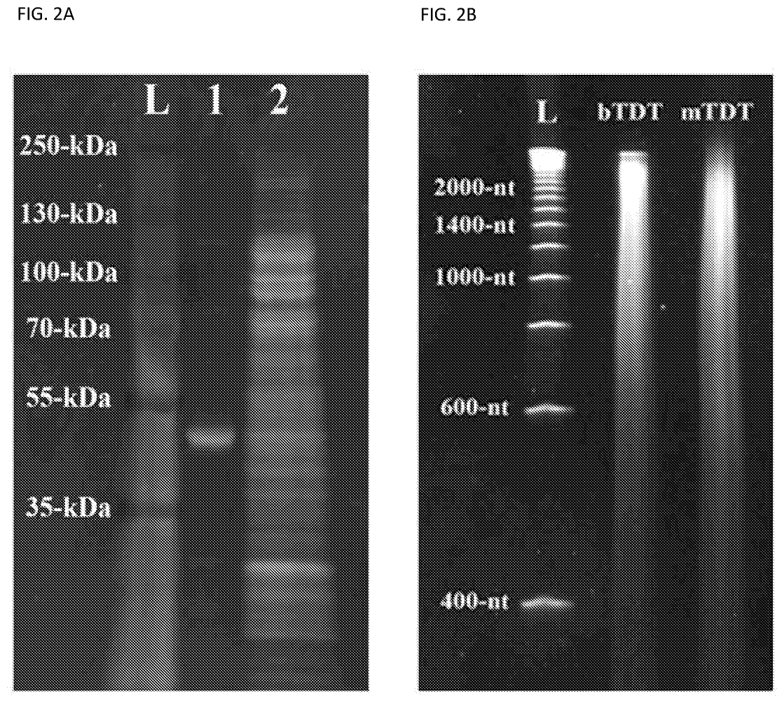

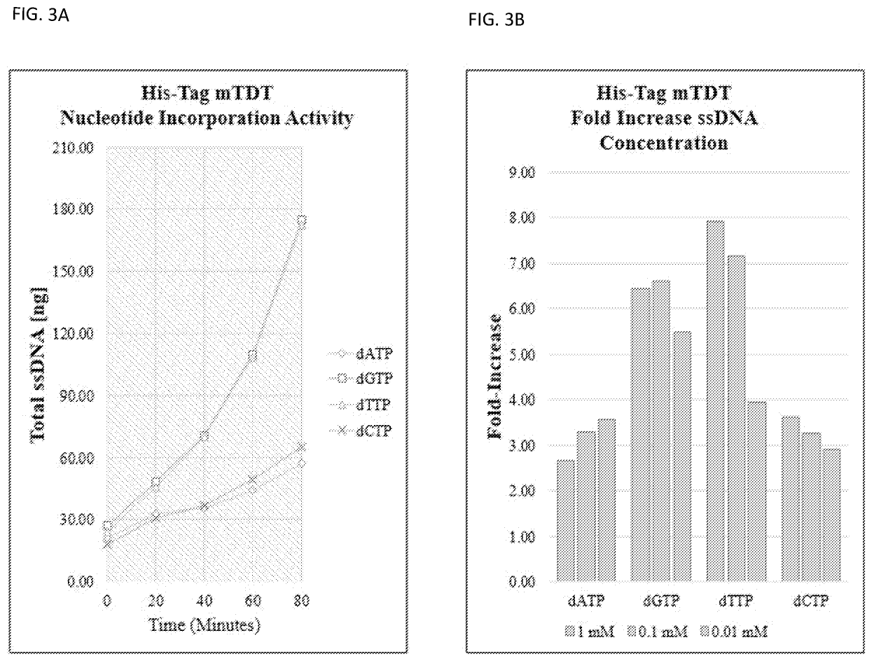

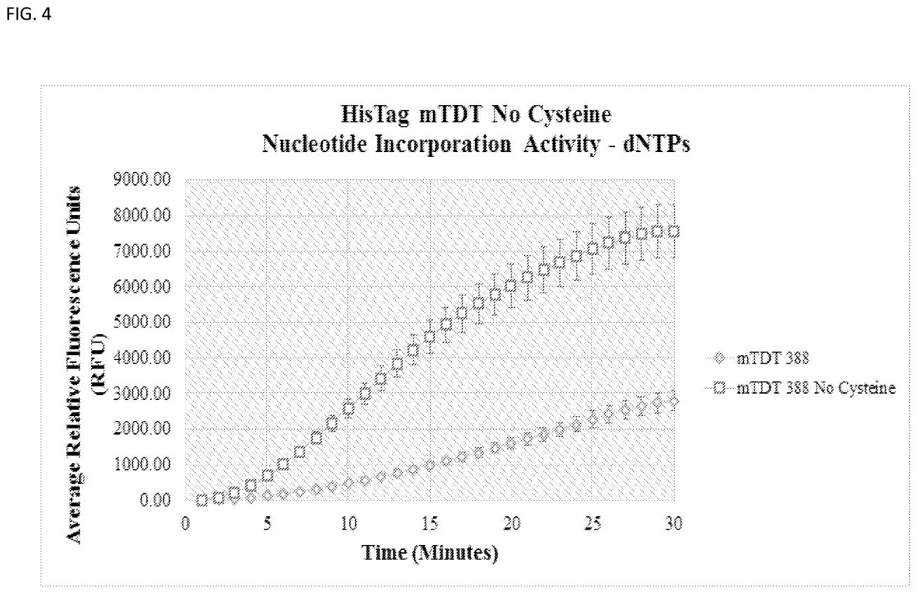

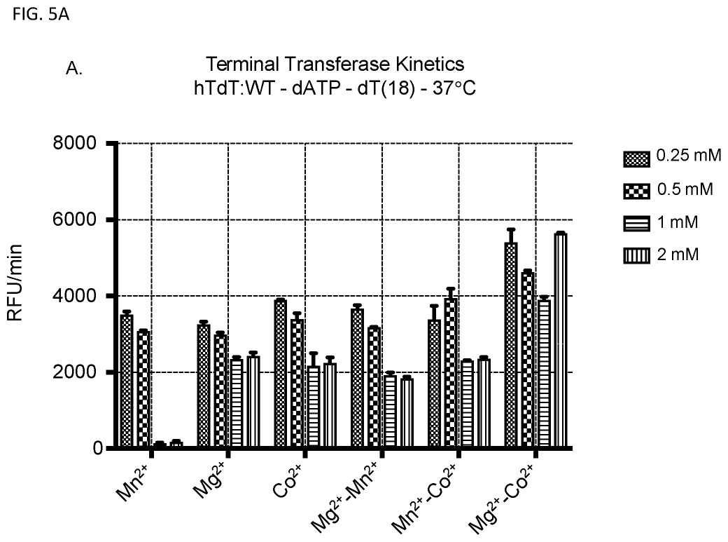

### DETAILED DESCRIPTION

Embodiments of the present disclosure are directed to methods of making a polynucleotide. It has been discovered that template-independent polymerase variants with the associated processivity factor can process the repeated addition of nucleotide molecules to the 3′ terminus of a single stranded target substrate oligonucleotide molecule under reaction conditions with improved processivity as compared to the template-independent polymerase alone or without the processivity factor. The processivity factor is associated with the template-independent polymerase in a manner to facilitate improved binding of the template-independent polymerase to a single stranded substrate. The processivity factor is attached to the template-independent polymerase via covalent or nonconvalent interactions. In some embodiments, linkers or binder pairs are included to join the processivity factor to the template-independent polymerase such that processing of the target substrate by the template-independent polymerase is improved. In other embodiments, the template-independent polymerase and the processivity factor are immobilized such as on a solid support in an orientation which facilitates processing of the substrate by the template-independent polymerase. In some embodiments, the template-independent polymerase variants include mutants where either one or more cysteine residues are replaced by one or more non-cysteine residues or vice versa. In some other embodiments, the template-independent polymerase variants include mutants where residues central to enzyme functionality, such as those located in the catalytic pocket, are replaced with different residues to improve processivity or overall functionality. In other embodiments, the template-independent polymerase variants include mutants having one or more surface accessible cysteine residues. In some embodiments, the processivity factor comprises a prokaryotic or eukaryotic single stranded DNA binding protein such as an E. coli single stranded DNA binding protein.

Embodiments of the present disclosure are further directed to macromolecules including a template-independent polymerase having a processivity factor attached thereto. In certain embodiments, automated characterization assays are used to determine the processivity of the macromolecules to identify candidates with enhanced processivity.

### Nucleic Acids and Nucleotides

As used herein, the terms “nucleic acid molecule,” “nucleic acid sequence,” “nucleic acid fragment” and “oligomer” are used interchangeably and are intended to include, but are not limited to, a polymeric form of nucleotides that may have various lengths, including either deoxyribonucleotides or ribonucleotides, or analogs thereof.

In general, the terms “nucleic acid molecule,” “nucleic acid sequence,” “nucleic acid fragment,” “oligonucleotide” and “polynucleotide” are used interchangeably and are intended to include, but not limited to, a polymeric form of nucleotides that may have various lengths, either deoxyribonucleotides (DNA) or ribonucleotides (RNA), or analogs thereof. An oligonucleotide is typically composed of a specific sequence of four nucleotide bases: adenine (A); cytosine (C); guanine (G); and thymine (T) (uracil (U) for thymine (T) when the polynucleotide is RNA). According to certain aspects, deoxynucleotide triphosphates (dNTPs, such as dATP, dCTP, dGTP, dTTP) may be used. According to certain aspects, ribonucleotide triphosphates (rNTPs, such as rATP, rCTP, rGTP, rUTP) may be used. According to certain aspects, ribonucleotide diphosphates (rNDPs) may be used.

The term “oligonucleotide sequence” or simply “sequence is the alphabetical representation of a polynucleotide molecule; alternatively, the term may be applied to the polynucleotide molecule itself. This alphabetical representation can be input into databases in a computer having a central processing unit and used for bioinformatics applications such as functional genomics and homology searching. Oligonucleotides may optionally include one or more non-standard nucleotide(s), nucleotide analog(s) and/or modified nucleotides. The present disclosure contemplates any deoxyribonucleotide or ribonucleotide and chemical variants thereof, such as methylated, hydroxymethylated or glycosylated forms of the bases, and the like. According to certain aspects, natural nucleotides are used in the methods of making the nucleic acids. Natural nucleotides lack chain terminating moieties. According to another aspect, the methods of making the nucleic acids described herein do not use terminating nucleic acids or otherwise lack terminating nucleic acids, such as reversible terminators known to those of skill in the art. The methods are performed in the absence of chain terminating nucleic acids or wherein the nucleic acids are other than chain terminating nucleic acids.

Examples of modified nucleotides include, but are not limited to diaminopurine, S2T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone. Nucleic acid molecules may also contain amine-modified groups, such as aminoallyl-dUTP (aa-dUTP) and aminohexhylacrylamide-dCTP (aha-dCTP) to allow covalent attachment of amine reactive moieties, such as N-hydroxy succinimide esters (NHS).

Modified nucleotide mono, di, tri phosphates and their synthesis methods have been described (Roy, B., Depaix, A., Périgaud, C., & Peyrottes, S, (2016), Recent Trends in Nucleotide Synthesis. Chemical Reviews, 116(14), 7854-7897), which is hereby incorporated by reference in its entirety.

Alternatives to standard DNA base pairs or RNA base pairs in the oligonucleotides of the present disclosure can provide higher density in bits per cubic mm, higher safety (resistant to accidental or purposeful synthesis of natural toxins), easier discrimination in photo-programmed polymerases, or lower secondary structure. Such alternative base pairs compatible with natural and mutant polymerases for de novo and/or amplification synthesis are described in Betz K, Malyshev D A, Lavergne T, Welte W, Diederichs K, Dwyer T J, Ordoukhanian P, Romesberg F E, Marx A (2012) KlenTaq polymerase replicates unnatural base pairs by inducing a Watson-Crick geometry, Nature Chem. Biol. 8:612-614; See Y J, Malyshev D A, Lavergne T, Ordoukhanian P, Romesberg F E. J Am Chem Soc. 2011 Dec. 14; 133(49):19878-88, Site-specific labeling of DNA and RNA using an efficiently replicated and transcribed class of unnatural base pairs; Switzer C Y, Moroney S E, Benner S A. (1993) Biochemistry. 32(39):10489-96. Enzymatic recognition of the base pair between isocytidine and isoguanosine; Yamashige R, Kimoto M, Takezawa Y, Sato A, Mitsui T, Yokoyama S, Hirao I. Nucleic Acids Res. 2012 March; 40(6):2793-806. Highly specific unnatural base pair systems as a third base pair for PCR amplification; and Yang Z, Chen F, Alvarado J B, Benner S A. J Am Chem Soc. 2011 Sep. 28; 133(38):15105-12, Amplification, mutation, and sequencing of a six-letter synthetic genetic system. Other non-standard nucleotides may be used such as described in Malyshev, D. A., et al., Nature, vol. 509, pp. 385-388 (15 May 2014), which are hereby incorporated by reference in their entireties.

### Polymerases

As used herein, the terms “processive” and “processivity” refer to an enzyme's ability to catalyze consecutive reactions without releasing a substrate molecule. For example, a DNA polymerase is processive if the polymerase makes multiple nucleotide incorporations, whether the same or different, before disassociation from a substrate such as a primer as those terms are known in the art of enzymatic synthesis.

According to an alternative embodiment of the present invention, polymerases are used to build nucleic acid molecules. According to one aspect, such nucleic acid molecules may represent information which is referred to herein as being recorded in the nucleic acid sequence or the nucleic acid is referred to herein as being storage media. Polymerases are enzymes that produce a nucleic acid sequence, for example, using DNA or RNA as a template. Polymerases that produce RNA polymers are known as RNA polymerases, while polymerases that produce DNA polymers are known as DNA polymerases. Polymerases that incorporate errors are known in the art and are referred to herein as an “error-prone polymerases”. Template independent polymerases may be error prone polymerases. Using an error-prone polymerase allows the incorporation of specific bases at precise locations of the DNA molecule. Error-prone polymerases will either accept a non-standard base, such as a reversible chain terminating base, or will incorporate a different nucleotide, such as a natural or unmodified nucleotide that is selectively provided during primer extension.

### Template Independent Polymerases

As used herein, template-independent polymerases, refer to polymerase enzymes which catalyze extension of polynucleotide substrate or primer strand with nucleotides in the absence of a polynucleotide template. Template independent polymerases where the polynucleotide substrate or primer is DNA are known as template independent DNA polymerases. Template independent polymerases where the polynucleotide substrate or primer is RNA are known as template independent RNA polymerases. Template independent polymerases may accept a broad range of nucleotide polyphosphate substrates. Template independent DNA polymerase are defined to include all enzymes with activity classified by the Enzyme commission number EC 2.7.7.31 (See, enzyme—ExPASy: SIB Bioinformatics Resource Portal, EC 2.7.7.31).

According to certain aspects of the invention the template independent DNA polymerase is a terminal deoxynucleotidyl transferase (TdT) of the polX family of DNA polymerases. TdT may also be referred to as DNA nucleotidylexotransferase, (DNTT) or simply terminal transferase. According to further aspects of the disclosure, TdT is of mammalian origin, for example, from bovine or murine sources. Further description of TdT is provided in Biochim Biophys Acta., May 2010; 1804(5): 1151-1166, hereby incorporated by reference in its entirety. TdT creates polynucleotide strands by catalyzing the addition of nucleotides to the 3′ terminus of a DNA molecule in the absence of a template. The preferred substrate of TdT is a 3′-overhang, but it can also add nucleotides to blunt or recessed 3′ ends. Cobalt is a cofactor, however the enzyme catalyzes reaction upon Mg²⁺, Zn²⁺, and Mn⁺ administration in vitro. Nucleic acid initiator fragments or sequences may be 4 or 5 nucleotides or longer and may be single stranded or double stranded. Double stranded initiators may have a 3′ overhang or they may be blunt ended or they may have a 3′ recessed end. Preferred nucleotides are dTTP, dATP, dGTP, dCTP. TdT can catalyze incorporation of many modified nucleotides.

According to certain aspects of the disclosure, the template independent DNA polymerase is a terminal deoxynucleotidyl transferase of the archaeo-eukaryotic primase (AEP) superfamily. Exemplary terminal transferases are described (Guilliam, T. A., Keen, B. A., Brissett, N. C., & Doherty, A. J, (2015), Primase-polymerases are a functionally diverse superfamily of replication and repair enzymes, Nucleic Acids Research, 43(14), 6651-64), which is hereby incorporated by reference in its entirety.

In further aspects of the disclosure, the terminal transferase is PolpTN2, a DNA primase-polymerase protein encoded by the pTN2 plasmid from Thermococcus nautilus. In further aspects of the contemplated disclosure a C-terminal truncation of PolpTN2 may be used, such as Δ₃₁₁₋₉₂₃. (Sukhvinder Gill et al., A highly divergent archaeo-eukaryotic primase from the Thermococcus nautilus plasmid, pTN2, Nucleic Acids Research, Volume 42, Issue 6, Pp. 3707-3719, http://doi.org/10.1093/nar/gkt1385)

In further aspects of the disclosure, the terminal transferase is PriS, a primase S subunit from the kingdom Archea. For example: DNA primase complex of p41-p46 or PriSL as described in the following:

Pyrococcus furiosus (Lidong Liu et al., The Archaeal DNA Primase Biochemical Characterization of the p41-p46 Complex from Pyrococcus Furiosus, The Journal of Biological Chemistry, 276, 45484-45490, 2001, doi:10.1074/jbc.M106391200),

Thermococcus kodakaraensis (Wiebke Chemnitz Galal et al., Characterization of DNA Primase Complex Isolated from the Archaeon, Thermococcus kodakaraensis, The Journal of Biological Chemistry 287, 16209-16219, 2012, doi: 10.1074/jbc.M111.338145),

Sulfolobus solfataricus (Si-houy Lao-Sirieix, et al., The Heterodimeric Primase of the Hyperthermophilic Archaeon Sulfolobus solfataricus Possesses DNA and RNA Primase, Polymerase and 3′-terminal Nucleotidyl Transferase Activities, Journal of Molecular Biology, Volume 344, Issue 5, 2004, Pages 1251-1263, http://dx.doi.org/10.1016/j.jmb.2004.10.018),

Pyrococcus horikoshii (Eriko Matsui et al., Distinct Domain Functions Regulating de Novo DNA Synthesis of Thermostable DNA Primase from Hyperthermophile Pyrococcus horikoshii, Biochemistry, 2003, 42 (50), pp 14968-14976, DOI: 10.1021/bi035556o), and

Archaeoglobus fulgidus (Stanislaw K. Jozwiakowski, et al., Archaeal replicative primases can perform translesion DNA synthesis, PNAS, 2015, vol. 112, no. 7, E633-E638, doi: 10.1073/pnas. 1412982112), which are hereby incorporated by reference in their entireties.

In further aspects of the disclosure, the terminal transferase is an archeal non-homologous end joining archaeo-eukaryotic primase.

In further aspects of the disclosure, the terminal transferase is a mammalian Pol θ as described in Tatiana Kent, et al., Mechanism of microhomology-mediated end-joining promoted by human DNA polymerase θ, Nature Structural & Molecular Biology, Vol. 22, 230-237, (2015), doi: 10.1038/nsmb.2961, hereby incorporated by reference in its entirety.

In further aspects of the disclosure, the terminal transferase is a Eukaryotic PrimPol, for example, human primPol have been described as in Sara Garcia-Gmez, et al., PrimPol, an Archaic Primase/Polymerase Operating in Human Cells, Molecular Cell, Volume 52, Issue 4, 2013, Pages 541-553, http://dx.doi.org/10.1016/j.molcel.2013.09.025, Thomas A. Guilliam, et al., Human PrimPol is a highly error-prone polymerase regulated by single-stranded DNA binding proteins, Nucl. Acids Res., (2015), 43 (2): 1056-1068, doi: 10.1093/nar/gkul321, which are hereby incorporated by reference in their entireties.

As is known in the art of using a template-independent polymerase to build nucleic acid sequences, TdT or other DNA polymerases, may require divalent metal ions for catalysis. However, TdT is unique in its ability to use a variety of divalent cations such as Co²⁺, Mn²⁺, Zn²⁺ and Mg²⁺. In general, the extension rate of the primer p(dA)n (where n is the chain length from 4 through 50) with dATP in the presence of divalent metal ions is ranked in the following order: Mg²⁺>Zn²⁺>Co²⁺>Mn²⁺. In addition, each metal ion has different effects on the kinetics of nucleotide incorporation. For example, Mg²⁺ facilitates the preferential utilization of dGTP and dATP whereas Co²⁺ increases the catalytic polymerization efficiency of the pyrimidines, dCTP and dTTP. Zn²⁺ behaves as a unique positive effector for TdT since reaction rates with Mg²⁺ are stimulated by the addition of micromolar quantities of Zn²⁺. This enhancement may reflect the ability of Zn²⁺ to induce conformational changes in TdT that yields higher catalytic efficiencies. Polymerization rates are lower in the presence of Mn²⁺ compared to Mg²⁺, suggesting that Mn²⁺ does not support the reaction as efficiently as Mg^{2+.}Further description of TdT is provided in Biochim Biophys Acta., May 2010; 1804(5): 1151-1166 hereby incorporated by reference in its entirety.

### Polymerase Processivity Factors:

As used herein, “polymerase processivity factor” and “processivity factor” are used interchangeably, and are defined to mean polypeptide domains and subdomains which confer sequence independent polynucleotide interactions, and are associated with a polymerase by covalent or noncovalent interactions.

Processivity factors are polypeptide domains or subdomains that confer enhanced processivity to an enzyme, such as a polymerase. In context of polymerases, they confer a lower dissociation constant between the polymerase and the polynucleotide substrate, allowing for more nucleotide incorporations on average before dissociation of the polymerase from the substrate or primer.

Polymerase processivity factors function by multiple sequence independent polynucleotide binding mechanisms and association to a polymerase. The primary mechanism is electrostatic interaction between the polynucleic acid phosphate backbone and the processivity factor. The second is steric interactions between the processivity factor with the minor groove structure of the duplex. The third mechanism, is topological restraint, where interactions with the polynucleotide are facilitated by clamp proteins that completely encircle the polynucleotide, with which they associate.

Exemplary sequence independent polynucleotide binding domains are known in the art, and are traditionally classified according to the preferred nucleic acid substrate, for example, DNA or RNA and strandedness, such as single stranded or double stranded. One embodiment of the disclosure considers single stranded DNA binding domains/proteins as processivity enhancing factors.

Various polypeptide domains have been identified as polynucleotide binders. These polypeptide domains include four general structural topologies known to bind ssDNA: oligonucleotide-binding (OB) folds, K homology (KH) domains, RNA recognition motifs (RRMs), and whirly domains as described in Thayne H. Dickey et al., Single-Stranded DNA-Binding Proteins: Multiple Domains for Multiple Functions, Structure, Volume 21, Issue 7, 2 Jul. 2013, Pages 1074-1084, http://dx.doi.org/10.1016/j.str.2013.05.013, hereby incorporated by reference in its entirety.

Oligonucleotide binding domains (OBDs) are exemplary DNA binding domains structurally conserved in multiple DNA processing proteins. OBDs bind with ssDNA ligands from 3 to 11 nucleotides per OB fold and dissociation constants ranging from low-picomolar to high-micromolar levels. Affinities roughly correlate with the length of ssDNA bound. Some OBDs may confer sequence specific binding, while others are non-sequence specific.

Exemplary OBD containing DNA-binding proteins specifically bind single-stranded DNA (ssDNA) are so called ‘single stranded DNA binding proteins’ (SSBs). SSB domains are well known to those of skill in the art, for example, as described in James L. Keck (ed.), Single-Stranded DNA Binding Proteins: Methods and Protocols, Methods in Molecular Biology Volume 922, pp 37-54, Springer Science+Business Media, LLC 2012, DOI 10.1007/978-1-62703-032-8_3 and Alexander G. Kozlov, et al., SSB Binding to ssDNA Using Isothermal Titration Calorimetry, Robert D. Shereda, et al., SSB as an Organizer/Mobilizer of Genome Maintenance Complexes, CRITICAL REVIEWS IN BIOCHEMISTRY AND MOLECULAR BIOLOGY, VOL. 43, ISS. 5, Pages 289-318, 2008, http://dx.doi.org/10.1080/10409230802341296, hereby incorporated by reference in their entireties. SSBs describe a family of evolved molecular chaperones of single stranded DNA. In vivo, SSBs serve multiple functions, for example stabilizing ssDNA at the replication fork, recombination and genome repair. SSBs may be composed of multiple subunits as pentameric, tetrameric, trimeric, dimeric or monomeric complexes. SSBs are exemplary processivity enhancing factors for single stranded DNA substrates, because of the ability to translocate across single stranded DNA, and the relatively large potential barrier from unbinding from the single stranded DNA substrate, and a lack of sequence specific binding preference.

Several exemplary prokaryotic SSBs have been characterized as known to those of skill in the art. These SSBs include, but are not limited to: Escherichia coli SSB (see Srinivasan Raghunathan et al., Nature Structural & Molecular Biology, Structure of the DNA binding domain of E. coli SSB bound to ssDNA, 7, 648-652 (2000), doi:10.1038/77943), Deinococcus radiodurans SSB (see Lockhart J S, DeVeaux LC (2013) The Essential Role of the Deinococcus radiodurans ssb Gene in Cell Survival and Radiation Tolerance. PLoS ONE 8(8):e71651, doi:10.1371/journal.pone.0071651), Sulfolobus solfataricus SSB (see Sonia Paytubi, et al., Displacement of the canonical single-stranded DNA-binding protein in the Thermoproteales, PNAS, 2012, vol. 109, no. 7, E398-E405, doi: 10.1073/pnas.1113277108), Tth SSB and Thermus aquaticus SSB (see Gregor Witte, et al., Biophysical Analysis of Thermus aquaticus Single-Stranded DNA Binding Protein, Biophysical Journal, Volume 94, Issue 6, 2008, Pages 2269-2279, http://dx.doi.org/10.1529/biophysj.107.121533), Deinococcus radiopugnans SSB (see Filipkowski, P., Koziatek, M. & Kur, J., A highly thermostable, homodimeric single-stranded DNA-binding protein from Deinococcus radiopugnans, Extremophiles, (2006), 10: 607, doi:10.1007/s00792-006-0011-8), hereby incorporated by reference in their entireties.

Escherichia coli SSB (EcSSB) is a well-studied eubacterial SSB. Structural characterization of the EcSSB-ssDNA complex revealed that EcSSB is a homotetrameric protein, and binds single stranded DNA in a similar geometry to seams on a baseball (See, Srinivasan Raghunathan, et al., Structure of the DNA binding domain of E. coli SSB bound to ssDNA, Nature Structural & Molecular Biology, 7, 648-652, (2000), doi:10.1038/77943, hereby incorporated by reference in its entirety). EcSSB has DNA binding footprint of approximately 65 nucleotides, EcSSB has 12 tryptophan residues colocalized with OBDs that contact the DNA every 3 bases. Tryptophan-DNA interactions are stabilized by a general Pi stacking mechanism. Based on single molecule force spectrometry studies, EcSSB is understood to diffuse on ssDNA using a reptation mechanism, that involves the diffusion of 3 bp defects through the complex as described in Ruobo Zhou, et al., SSB Functions as a Sliding Platform that Migrates on DNA via Reptation, Cell, 2011, Volume 146, Issue 3, p485, DOI: http://dx.doi.org/10.1016/j.cell.2011.07.027, hereby incorporated by reference in its entirety.

In non-eubacterial systems, functional eukaryotic homologs to the bacterial SSB protein family are known to those of skill in the art. Replication protein A (RPA) is an exemplary homolog used in DNA replication, recombination and DNA repair in eukaryotes. The RPA heterotrimer is comprised of RPA70, RPA32, RPA14 subunits as described in Cristina Iftode, et al., Replication Protein A (RPA): The Eukaryotic SSB, Critical Reviews in Biochemistry and Molecular Biology, 2008, Volume 34, 1999—Issue 3, Pages 141-180, DOI: http://dx.doi.org/10.1080/10409239991209255, hereby incorporated by reference in its entirety.

### Attachment of Polymerase Processivity Factors to Template Independent Polymerases

As used herein, the term “attach,” or “attachment” refers to both covalent interactions and noncovalent interactions. A covalent interaction is a chemical linkage between two atoms or radicals formed by the sharing of a pair of electrons (i.e., a single bond), two pairs of electrons (i.e., a double bond) or three pairs of electrons (i.e., a triple bond). Covalent interactions are also known in the art as electron pair interactions or electron pair bonds. Noncovalent interactions include, but are not limited to, van der Waals interactions, hydrogen bonds, weak chemical bonds (i.e., via short-range noncovalent forces), hydrophobic interactions, ionic bonds and the like. A review of noncovalent interactions can be found in Alberts et al., in Molecular Biology of the Cell, 3d edition, Garland Publishing, 1994, hereby incorporated by reference in its entirety.

Suitable polymerase processivity factors will bind the polynucleotide substrate with affinities in excess of the template independent polymerase alone. Presupposing the processivity factor affinity is sufficiently large, the limit of polymerase processivity will be governed by affinity of the processivity factor to the polymerase. The contemplated disclosure comprises of a number of methods for attachment of a processivity factor to a template Independent DNA polymerase.

In certain exemplary embodiments of covalent attachment, the processivity factor and the template independent DNA polymerase are constructed as a protein fusion. The terms “protein fusion” and “peptide fusion” are used interchangeably and are defined as the concatenation of two peptide sequence of two or more polypeptide domain sequences. In certain embodiments, the protein fusion may be comprised of an insertion of a polypeptide sequence of a polypeptide domain within the polypeptide sequence of another polypeptide domain, referred to herein as an interdomain fusion. The term ‘linker sequence’, as used herein, refers to a polypeptide sequence with certain properties. A linker sequence may be stiff or flexible, depending on the application. In certain embodiments of protein fusions, a ‘linker’ polypeptide sequence may be introduced between two polypeptide sequence to provide extra distance and flexibility or rigidity between protein domains and subdomains. Various compositions of linker sequences are known to those of skill in the art (See, e.g., Xiaoying Chen, et al., Fusion protein linkers: Property, design and functionality, Advanced Drug Delivery Reviews, 2013, Volume 65, Issue 10, Pages 1357-1369, DOI: http://dx.doi.org/10.1016/j.addr.2012.09.039, hereby incorporated by reference in its entirety).

In yet other embodiments of covalent attachment, one or more of the polypeptide sequences are circular permuted prior to protein fusion. Circular permutation is practiced by those of skill in the art (See, e.g., Ying Yu and Stefan Lutz, Circular permutation: a different way to engineer enzyme structure and function, Trends in Biotechnology, 2011, Volume 29, Issue 1, Pages 18-25, DOI: http://dx.doi.org/10.1016/j.tibtech.2010.10.004, hereby incorporated by reference in its entirety). In the art of protein engineering, it is common to introduce a polypeptide linker sequence between the N and C terminus of the polypeptide, prior to circular permutation, which entails introducing new termini in a protein by cleavage of an existing peptide bond. Circular Permutation introduces novel positions on a protein for fusion to alternative domains or subdomains with respect to local tertiary structure.

In alternative embodiments of covalent attachment, the processivity factor and template independent polymerase domains are chemically crosslinked by a polyfunctional molecule containing a plurality of protein reactive moieties. Protein crosslinking is known to those of skill in the art as a form of protein conjugation. A review of the art of protein conjugation is hereby incorporated by reference in its entirety (see Hermanson, G. T., (2013), Bioconjugate Techniques. Academic Press). In one embodiment, bifunctional cross linker may comprise of the homobifunctional moieties or heterobifunctional moieties, and other reactive groups compatible with polypeptide side chains suitable for monovalent functionalization.

Monovalent Chemical Protein Conjugation

| Crosslinking target | Reactive groups |
|---|---|
| Amine-to-amine | Di NHS esters/di isocyanates/di isothiocyanates/di |
| | acid halide/di anhydride. |
| | For example: Bis[2-(N-succinimidyl- |
| | oxycarbonyloxy)ethyl]sulfone, Di(N-succinimidyl) glutarate, |
| | Sebacic acid bis(N-succinimidyl) ester, p-Phenylene |
| | diisothiocyanate, |
| | 4,7,10,13,16,19,22,25,32,35,38,41,44,47,50,53-Hexadecaoxa- |
| | 28,29-dithiahexapentacontanedioic acid di-N-succinimidyl |
| | ester, DTSSP (3,3′-dithiobis(sulfosuccinimidyl propionate)), |
| | Sulfo-EGS (ethylene glycol bis(sulfosuccinimidyl succinate)), |
| | DST (disuccinimidyl tartrate), BS(PEG)9 (PEGylated |
| | bis(sulfosuccinimidyl)suberate), BS(PEG)5 (PEGylated |
| | bis(sulfosuccinimidyl)suberate), Dimethyl 3,3′- |
| | dithiopropionimidate, 4,4′-Diisothiocyanatostilbene-2,2′- |
| | disulfonic acid, 3,3′-Dithiodipropionic acid di(N- |
| | hydroxysuccinimide ester), Dimethyl pimelimidate |
| | dihydrochloride, Ethylene glycol-bis(succinic acid N- |
| | hydroxysuccinimide ester), Suberic acid bis(N- |
| | hydroxysuccinimide ester), Suberic acid bis(3-sulfo-N- |
| | hydroxysuccinimide ester), and the like. |
| Sulfhydryl-to- | Di Maleimides/di haloacetyl/di pyridyldithiol/di |
| sulfhydryl | vinylsulfone/di alkene with radical. For example: 1,4-Bis[3- |
| | (2-pyridyldithio)propionamido]butane, BMOE (bis- |
| | maleimidoethane), BM(PEG)2 (1,8-bismaleimido- |
| | diethyleneglycol), BM(PEG)3 (1,11-bismaleimido- |
| | triethyleneglycol), DTME (dithio-bis-maleimidoethane), and |
| | the like. |
| Amine to sulfhydryl | (NHS ester, isocynanate, isothiocyanate, acid halid, or |
| | anhydride) and (maleimide, haloacetyl, pyridyldithiol, |
| | vinylsulfone, or alkene with radical) For Example: Sulfo- |
| | SMPB (sulfosuccinimidyl 4-(N-maleimidophenyl)butyrate), |
| | Sulfo-SIAB (sulfosuccinimidyl (4-iodoacetyl)aminobenzoate), |
| | Sulfo-N-succinimidyl 4-maleimidobutyrate, Sulfo-MBS (m- |
| | maleimidobenzoyl-N-hydroxysulfosuccinimide ester), Sulfo- |
| | LC-SPDP (sulfosuccinimidyl 6-[3′-(2- |
| | pyridyldithio)propionamido]hexanoate), Sulfo-KMUS (N-(κ- |
| | maleimidoundecanoyloxy) sulfosuccinimide ester), Sulfo- |
| | EMCS (N-(ε-maleimidocaproyloxy) sulfosuccinimide ester), |
| | SMPT (4-succinimidyloxycarbonyl-alpha-methyl-alpha(2- |
| | pyridyldithio)toluene), SMPH (succinimidyl-6-((b- |
| | maleimidopropionamido)hexanoate), SM(PEG)24 |
| | (PEGylated, long-chain SMCC crosslinker), SIAB (N- |
| | succinimidyl (4-iodoacetyl)aminobenzoate), SBAP |
| | (succinimidyl 3-(bromoacetamido)propionate), PEG4-SPDP |
| | (PEGylated, long-chain SPDP crosslinker), PEG12-SPDP |
| | (PEGylated, long-chain SPDP crosslinker), O—[N-(3- |
| | Maleimidopropionyl)aminoethyl]-O′-[3-(N-succinimidyloxy)- |
| | 3-oxopropyl]triethylene glycol, O—[N-(3- |
| | Maleimidopropionyl)aminoethyl]-O′-[3-(N-succinimidyloxy)- |
| | 3-oxopropyl]heptacosaethylene glycol, Maleimidoacetic acid |
| | N-hydroxysuccinimide ester, Maleimide-PEG8-succinimidyl |
| | ester, Maleimide-PEG6-succinimidyl ester, Maleimide-PEG2- |
| | succinimidyl ester, Maleimide-PEG12-succinimidyl ester, LC- |
| | SPDP (succinimidyl 6-[3(2- |
| | pyridyldithio)propionamido]hexanoate), LC-SMCC |
| | (succinimidyl-4-(N-maleimidomethyl)cyclohexane-1- |
| | carboxy-(6-amidocaproate)), Iodoacetic acid N- |
| | hydroxysuccinimide ester, Bromoacetic acid N- |
| | hydroxysuccinimide ester, 6-Maleimidohexanoic acid N- |
| | hydroxysuccinimide ester, 4-Maleimidobutyric acid N- |
| | hydroxysuccinimide ester, 4-(4-Maleimidophenyl)butyric |
| | acid N-hydroxysuccinimide ester, 3-Maleimidopropionic acid |
| | N-hydroxysuccinimide ester, 3-(2-Pyridyldithio)propionic |
| | acid N-hydroxysuccinimide ester, and the like. |
| Carboxyl-to-amine | Carbodiimide |
| Hydroxyl-to-sulfhydryl | Isocyanate and (maleimide, haloacetyl, pyridyldithiol, |
| | vinylsulfone, or alkene with radical) |

| Protein Group | Crosslinking Group |
|---|---|
| Cysteine | Maleimide, vinyl sulfone, haloacetyl, |
| | pyridyldithiol, thioester (only works for |
| | N-terminal cysteine) |
| Biotin | Streptavidin, Avidin |
| Unnatural amino Acid (Azide) | Alkyne, cyclooctyne |
| Unnatural amino Acid (Alkyne) | Azide |

In alternative embodiments, the attachment of the processivity factor to the template independent polymerase is facilitated by a non-covalent interaction. Non-covalent attachment may be facilitated by intrinsic binding affinity between the processivity factor and the template independent DNA polymerase. As used herein, “contact” in reference to weak non-covalent chemical interactions, such as van der Waal forces, hydrogen bonding, base-stacking interactions, ionic and hydrophobic interactions, and the like, dominate the interaction of the molecules.

Non-covalent attachment may also be facilitated by addition of a protein binding domain with high affinity to the complement domain, for example by protein fusion of a processivity factor binding domain to a template independent DNA polymerase, or by fusion of a template independent DNA polymerase binding domain to a processivity factor. As used herein, “binding domain” may comprise of the following classes of protein binders.

| Protein | |
|---|---|
| Binder | Scaffold |
| Antibody | Fv Domain from IgG |
| Single-chain | Fv Domain from IgG |
| variable | |
| fragment | |
| Single | Variable domain from IgG of the Camelid Family, Variable |
| Domain | domain from IgG Heavy chain in Carteleginic Fish |
| Antibody | |
| Affibodies | Z domain of Protein A |
| Affilins | Gamma-B crystalline, Ubiquitin |
| Affimers | Cystatin |
| Affitins | Sac7d (from Sulfolobus acidocaldarius) |
| Alphabodies | Triple helix coiled coil |
| Anticalins | Lipocalins |
| Avimers | A domains of various membrane receptors |
| DARPins | Ankyrin repeat motif |
| Fynomers | SH3 domain of Fyn |
| Monobodies | 10th type III domain of fibronectin |

Non-covalent attachment may also be facilitated by protein fusion of interacting protein binding domains with mutual affinity to the processivity factor, and template independent DNA polymerase. In an additional aspect, a polymerase binding domain is attached to a processivity factor. In an additional aspect, a processivity binding domain is attached to a polymerase. As used herein, “binding domain” may comprise of synthetic coiled-coil domains or classes of mutually interacting proteins.

Non-covalent attachment may also be facilitated by colocalized attachment of both a Templated independent DNA polymerase and a processivity factor to a common surface or support. The term “colocalized” as used in reference to proteins, is defined to mean that two proteins are within a mutual radius of gyration from their respective surface attachment positions. In this embodiment, “colocalization” will mean the optimal spacing and orientation of processivity factor and polymerase to confer maximum stability of bound substrate by the polymerase.

The processivity factor may be comprised of more than 1 subunit, in which case, it is advantageous to construct a protein fusion comprised of the required subunits as a single multidomain protein. Monomeric proteins greatly simplify purification and design of functional complexes in protein engineering, however they are not required to ensure the functionality of a system processivity factors and template independent DNA polymerases.

### Supports and Attachment

In certain exemplary embodiments, one or more oligonucleotide sequences, such as substrates or primers as described herein may be immobilized on a support (e.g., a solid and/or semi-solid support). Alternatively, polymerases and processivity factors as described herein may be attached to a support. In certain aspects, an oligonucleotide sequence can be attached to a support using one or more of the phosphoramidite linkers described herein. Polypeptide sequences may also be bound to substrates using methods and linkers (cleavable or non cleavable) and chemistry known to those of skill in the art. Suitable supports include, but are not limited to, slides, beads, chips, particles, strands, gels, sheets, tubing, spheres, containers, capillaries, pads, slices, films, plates and the like. In various embodiments, a solid support may be biological, nonbiological, organic, inorganic, or any combination thereof. Supports of the present invention can be any shape, size, or geometry as desired. For example, the support may be square, rectangular, round, flat, planar, circular, tubular, spherical, and the like. When using a support that is substantially planar, the support may be physically separated into regions, for example, with trenches, grooves, wells, or chemical barriers (e.g., hydrophobic coatings, etc.). Supports may be made from glass (silicon dioxide), metal, ceramic, polymer or other materials known to those of skill in the art. Supports may be a solid, semi-solid, elastomer or gel.

### Reagent Delivery Systems

According to certain aspects, reagents and washes are delivered at a desired location for a desired period of time to, for example, covalently attached dNTP to an initiator sequence or an existing nucleotide attached at the desired location using a template-independent polymerase as is known in the art. A selected nucleotide reagent liquid is deposited at the reaction site in the presence of a substrate as describe herein, one or more cations as described herein, a template-independent polymerase and a processivity factor as described herein where reaction takes place to add the dNTP to the substrate and then may be optionally followed by delivery of a buffer or wash that does not include the nucleotide. Suitable delivery systems include fluidics systems, microfluidics systems, syringe systems, ink jet systems, pipette systems and other fluid delivery systems known to those of skill in the art. Various flow cell embodiments or flow channel embodiments or microfluidic channel embodiments are envisioned which can deliver separate reagents or a mixture of reagents or washes using pumps or electrodes or other methods known to those of skill in the art of moving fluids through channels or microfluidic channels through one or more channels to a reaction region or vessel where the surface of the substrate is positioned so that the reagents can contact the desired location where a nucleotide is to be added.

According to another embodiment, a microfluidic device is provided with one or more reservoirs which include one or more reagents which are then transferred via microchannels to a reaction zone where the reagents are mixed and the reaction occurs. Such microfluidic devices and the methods of moving fluid reagents through such microfluidic devices are known to those of skill in the art.

### Protein Expression

The terms ‘expression’ or simply ‘protein expression’, refer to the generation of a recombinant protein product of interest in excess. Methods of protein expression are well known in the art (See, e.g., Protein production and purification, Nat Methods., 2008, 5(2): 135-146, doi: 10.1038/nmeth.f.202/, hereby incorporated by reference in its entirety). Commonly, a bacterial host is employed, such as Escherichia coli BL21(DE3 strains). Alternatively the host may be eukaryotic, for example, the yeasts Pichia pastoris described in (Mewes Boettner, et al., High-throughput screening for expression of heterologous proteins in the yeast Pichia pastoris, Journal of Biotechnology, 2002, Volume 99, Issue 1, Pages 51-62, DOI: http://dx.doi.org/10.1016/S0168-1656(02)00157-8, hereby incorporated by reference in its entirety) and Saccharomyces cerevisiae described in (Caterina Holz, et al., A micro-scale process for high-throughput expression of cDNAs in the yeast Saccharomyces cerevisiae, Protein Expression and Purification, 2002, Volume 25, Issue 3, Pages 372-378, DOI: http://dx.doi.org/10.1016/S1046-5928(02)00029-3, hereby incorporated by reference in its entirety), human cells (A. R. Aricescu, W. Lu and E. Y. Jones, A time- and cost-efficient system for high-level protein production in mammalian cells, Acta Cryst., (2006), D62, 1243-1250, DOI: https://doi.org/10.1107/S0907444906029799, hereby incorporated by reference in its entirety), or cell-free systems using prokaryotic (Corinna Tuckey, et al., UNIT 16.31 Protein Synthesis Using a Reconstituted Cell-Free System, Current Protocols in Molecular Biology, 2014, DOI: 10.1002/0471142727.mb1631s108, hereby incorporated by reference in its entirety) or eukaryotic extracts.

### Protein Purification

The terms “protein purification”, when used herein in reference to polypeptides, attached polypeptides, or proteins is defined as a process separation of a subset of molecules from a mixture of molecules. Protein purification methods are well known in the art. See, e.g., R. K. Scopes, Protein Purification: Principles and Practice (Springer Advanced Texts in Chemistry), Springer, 1994, hereby incorporated by reference in its entirety. Exemplary processes of protein purification include affinity chromatography, size exclusion chromatography, ion exchange chromatography and the like. In purifications involving affinity chromatography, it is common to use a resin, comprising of functional moieties with selective affinity to a protein affinity tag.

As used herein, the terms ‘protein affinity tag’, or simply ‘protein tag’ are defined as a polypeptide sequences introduced at the terminal ends of a protein or internal to the protein sequence which confers additional affinity to a substrate. Protein affinity tags and their use are known to those of skill in the art, see, e.g., Fujita-Yamaguchi Yoko, Affinity Chromatography of Native and Recombinant Proteins from Receptors for Insulin and IGF-I to Recombinant Single Chain Antibodies, Frontiers in Endocrinology, 2015, Vol. 6, pages 166, DOI: 10.3389/fendo.2015.00166, hereby incorporated by reference in its entirety. Common exemplary protein affinity tags include His-tags, comprised of 5-10 histidines that bind nickel or cobalt chelate, or Glutathione-S-transferase (GST) tags, a protein which binds to immobilized glutathione. In certain application of affinity purification proteins containing affinity tags may be eluted from the resin by competitive binding of a ligand, for example imidazole for release of His-tagged proteins from Ni(II)-nitrilotriacetic acid (Ni-NTA) resins. In other applications of protein purification, the protein may be cleaved from the resin by cleavage of a terminal protein tag. For example, it is common to introduce a protease recognition sequence such as a TEV protease recognition sequence between the protein and a terminal affinity tag such as GST tag to enable protease cleavage of protein from the GST resin.

### Template Independent DNA Polymerase Functional Assay

The terms “enzymatic activity” or “enzyme kinetics” are used herein to describe a protein's ability to catalyze a chemical reaction and is defined by a standardized unit of measure which is based on the rate at which reaction products are formed or the rate in which a reaction is completed. This rate is commonly defined as a “rate unit”, which is further defined by the amount of protein needed to efficiently catalyze a reaction in order to accomplish its intended function over a period of time under a predefined set of reaction conditions (See, e.g., Berg, Jeremy M., John L. Tymoczko, and Lubert Stryer. Biochemistry. New York: W.H. Freeman, 2002, hereby incorporated by reference in its entirety). For example, the rate unit of template independent polymerases such as a TdT could be described by the amount of protein that is needed to catalyze the incorporation of a certain concentration of natural or non-natural nucleotides into a single-stranded polynucleotide sequence using an initiator strand that exists in-solution or bound to a surface. The term “kinetic assay” and “activity screen” is used herein to describe an experimental procedure or set of procedures that has the ability to evaluate the enzymatic activity and enzyme kinetics of a protein. The terms “multiplex” and “high-throughput” are used to describe the parallelizable nature of a kinetic assay by having the ability to determine the individual enzymatic activity of less than, equal to or greater than 96 protein variants and/or reaction conditions in a single experiment.

In an exemplary multiplex kinetic assay, the activity of multiple purified template independent DNA polymerase variants or complexes can be determined by the rate at which long single-stranded polynucleotide sequences are produced by measuring the fluorescent response in Relative Fluorescence Units (RFU) of a nucleic acid stain that is highly specific for single-stranded DNA. The accuracy of the kinetic assay is characterized by a minimal observable fluorescent response if double stranded DNA contaminants are present in the reaction vessel or if single-stranded polynucleotide sequences form unintended secondary structures such as hairpins, stem-loop structures or G-quadraplexes and the like. Terminal deoxynucleotidyl transferase activity is only present if an observable increase in fluorescent signal occurs in comparison to a negative control consisting of only initiator strand, free nucleotides, cofactor and appropriate buffers. In addition, a positive control consisting of commercially available terminal transferase, such as bovine terminal deoxynucleotidyl transferase (New England Biolabs, Inc.) may also be used to relatively gauge the activity of purified template independent DNA polymerase variants or complexes. Single stranded nucleic acid fluorescent stains suitable for kinetic assays are known to those of skill in the art and are described in (ThermoFisher Scientific Inc., The Molecular Probes Handbook, Nucleic Acid Detection and Analysis-Chapter 8, Nucleic Acid Stains-Section 8.1, hereby incorporated by reference in its entirety).

To further quantitate the rate unit of each individual purified template independent DNA polymerase variants, a concentration curve consisting of a single polynucleotide sequence greater than 10 nucleotides can be generated to yield a set of standardized fluorescent signals. Because the fluorescent response in the presence of TdT activity is directly correlated to the amount of single stranded polynucleotide present at a given reaction time interval, the exact amount of polynucleotide in terms of mass can be interpolated from the concentration versus RFU curve and tracked throughout the progression of the reaction. This produces a rate unit for a particular amount of protein in terms of “mass increase in single-stranded polynucleotide per minute”. The rate unit for this kinetic assay can be further quantitated given additional reaction parameters such as free nucleotide composition, cofactors, and initiator sequence composition as well as each component's respective concentration. This kinetic assay provides a highly accurate and standardized method to specifically determine the best-candidate TdT variants or complex in a cost-efficient and high-throughput activity screen.

### Processivity Factor Functional Assay

A processivity factor can be functionally assessed for DNA binding activity by methods known to those of skill in the art. In an exemplary assay, termed DNAse footprinting, individual purified processivity factors and their cognate ssDNA or dsDNA are incubated together at physiological conditions and subsequently processed by a nuclease, for example a DNA endonuclease and/or a DNA exonuclease. Functional processivity factor DNA binding is indicated by an upward, higher molecular weight band shift of the DNA substrate incubated with processivity factor as compared to a control or reference sample under electrophoretic separation. In certain aspects of DNAse footprinting, the negative control sample is a nuclease incubation of substrate DNA where no processivity factor is present. In other aspects of DNAse footprinting, a reference sample is where DNA substrate is used as is with processing. Further aspects of DNAse footprinting known in the art are described in Michael Brenowitz, et al., UNIT 12.4, DNase I Footprint Analysis of Protein-DNA Binding, Current Protocols in Molecular Biology, 2001, DOI: 10.1002/0471142727.mb1204s07, hereby incorporated by reference in its entirety.

### Processivity Assay

Methods for determining processivity of DNA polymerases are also known in the art. Processivity assays can assess the extent of processive ssDNA primer extension by processive template independent DNA polymerase variants or complexes. In an exemplary method, template independent DNA polymerase processivity is determined on ssDNA substrate by allowing the polymerase to extend the ssDNA for an initial period followed by the addition of a greater than 100-fold excess of dideoxy ssDNA which sequesters the polymerase if it dissociates from the initial ssDNA during the reaction. Alternative methods have been described in the art. See Tatiana Kent, et al., Polymerase 0 is a robust terminal transferase that oscillates between three different mechanisms during end-joining, eLife 2016; 5:e13740, DOI: http://dx.doi.org/10.7554/eLife.13740, hereby incorporated by reference in its entirety.

The following examples are set forth as being representative of the present disclosure. These examples are not to be construed as limiting the scope of the present disclosure as these and other equivalent embodiments will be apparent in view of the present disclosure, figures and accompanying claims.

### Example I

A template independent DNA polymerase derived from Mus Muluscus terminal deoxynucleotidyl transferase, (mTdT) is covalently attached to a monomeric single stranded binding protein, derived from Escherichia coli (mEcSSB). This mTdT is mTdT 388 which is truncated and lacks the BRCT1 domain common to many Family X polymerases and has been shown to be not critical to retaining terminal transferase activity. Additionally, the removal of this domain could additivity improve enzyme functionality and processivity with the attachment of the single stranded binding protein. Template-independent DNA polymerases derived from other mammalian or non-mammalian species could be also acceptable for attachment to the single-stranded binding protein. Template-independent DNA polymerases could also be the full-length wild-type enzyme.

A monomeric E. coli SSB (mEcSSB) was previously generated and characterized for functional studies in vivo and in vitro (See, Edwin Antony, et al., Multiple C-Terminal Tails within a Single E. coli SSB Homotetramer Coordinate DNA Replication and Repair, Journal of Molecular Biology, 2013, Volume 425, Issue 23, Pages 4802-4819, DOI: http://dx.doi.org/10.1016/j.jmb.2013.08.021, hereby incorporated by reference in its entirety).

At ion concentrations optimal for catalytic activity of mTdT and other template independent DNA polymerases, the ssDNA binding footprint of mEcSSB is 65 nucleotides in length and has a measured Kd of less than 1 picomolar. mEcSSB is an exemplary processivity domain for template independent extension of ssDNA because it permits active translocation of ssDNA about the SSB by a reptation mechanism, ensuring continuous contact with ssDNA product while tethered to an active DNA polymerase. Further, mEcSSB permits exceptionally high affinity to ssDNA such that dissociation of ssDNA from the template independent DNA polymerase is negligible, or nonexistent.

In one embodiment, the method of attachment of the DNA polymerase to the SSB is by sulfhydryl crosslinking. In another embodiment, monovalent cysteine conjugation of the SSB and DNA polymerase is ensured by engineering each protein to contain at most one surface accessible cysteine each.

In an exemplary cysteine modified mEcSSB, a solvent accessible ‘GGSC’ sequence is introduced at the C-terminal tail. In further embodiments, an N-terminal Tev cleavable GST tag is added to the mEcSSB to support affinity purification. In additional exemplary embodiments, the GST-tag mEcSSB is modified by the following mutations (C85S, C138S, C169S, C178S) to remove all cysteines.

In an exemplary cysteine modified mTdT, all 7 native cysteine side chains in the short murine TdT (mTdT) 388 amino acid isoform (coordinates reference PDB ID:4I27) were replaced by non-cysteine residues. In further embodiments, the mTdT may contain an N-terminal His-tag for purification. In further embodiments of the mutant mTdT, the following mutations can be selected for cysteine knockout mutagenesis (C155A, C188A, C216N, C302A, C378G, C404A, and C438A). In other embodiments of mTdT388 cysteine knockout, additional cysteine knockout mutations were proposed as suitable alternatives to the exemplary set of mutations. In further embodiments, the following mutations may be used in various combinations for each cysteine position (C155A, C155S, C188A, C188G, C216A, C216D, C216G, C216N, C302A, C302G, C302P, C378A, C378G, C404A, C404S, C438A, and C438V). In further embodiments of mTdT388 cysteine knockout, the mutant may contain one of the following exemplary combinations of mutations: (C155A, C188A, C216N, C302P, C378A, C400A, C434A), (C155A, C188A, C216N, C302L, C378G, C400A, C434A), (C155A, C188M, C216N, C302L, C378G, C400A, C434A), (C155A, C188A, C216N, C302P, C378G, C400A, C434A), (C155A, C188A, C216N, C302M, C378G, C400A, C434A), (C155A, C188M, C216N, C302P, C378G, C400A, C434A).

In other aspects of cysteine engineered mTdT388, a single cysteine residue is reintroduced at a surface accessible region proximal to the primer binding site of the enzyme. In further aspects, cysteines may be introduced at positions that are proximal to the primer binding site, which include but are not limited to the following positions: (265,268,284,287), or certain other residues in the range of 260-280.

An exemplary mEcSSB sequence:

GST-TEV-mECSSB-MonoCys
(SEQ ID NO. 1)
MSPILGYWKIKGLVQPTRLLLEYLEEKYEEHLYERDEGDKWRNKKFELG
LEFPNLPYYIDGDVKLTQSMAIIRYIADKHNMLGGSPKERAEISMLEGA
VLDIRYGVSRIAYSKDFETLKVDFLSKLPEMLKMFEDRLSHKTYLNGDH
VTHPDFMLYDALDVVLYMDPMSLDAFPKLVSFKKRIEAIPQIDKYLKSS
KYIAWPLQGWQATFGGGDHPPKSDLVPRGSPEFPGRLERPHRDGGGSEN
LYFQGSGGASRGVNKVILVGNLGQDPEVRYMPNGGAVANITLATSESWR
DKATGEMKEQTEWHRVVLFGKLAEVASEYLRKGSQVYIEGQLRTRKWTD
QSGQDRYTTEVVVNVGGTMQMLQLGTQPELIQDAGGGVRMSGAGTASRG
VNKVILVGNLGQDPEVRYMPNGGAVANITLATSESWRDKATGEMKEQTE
WHRVVLFGKLAEVASEYLRKGSQVYIEGQLRTRKWTDQSGQDRYTTEVV
VNVGGTMQMLASHMASRGVNKVILVGNLGQDPEVRYMPNGGAVANITLA
TSESWRDKATGEMKEQTEWHRVVLFGKLAEVASEYLRKGSQVYIEGQLR
TRKWTDQSGQDRYTTEVVVNVGGTMQMLQLGTQPELIQDAGGGVRMSGA
GTASRGVNKVILVGNLGQDPEVRYMPNGGAVANITLATSESWRDKATGE
MKEQTEWHRVVLFGKLAEVASEYLRKGSQVYIEGQLRTRKWTDQSGQDR
YTTEVVVNVGGTMQMLGGSC*

An exemplary mid sequence without cysteine:

6xHis-mTdT388 NoCys
(SEQ ID NO. 2)
MGSSHHHHHHSSGLVPRGSHMSPSPVPGSQNVPAPAVKKISQYAAQRRT
TLNNYNQLFTDALDILAENDELRENEGSALAFMRASSVLKSLPFPITSM
KDTEGIPNLGDKVKSIIEGIIEDGESSEAKAVLNDERYKSFKLFTSVFG
VGLKTAEKWFRMGFRTLSKIQSDKSLRFTQMQKAGFLYYEDLVSAVNRP
EAEAVSMLVKEAVVTFLPDALVTMTGGFRRGKMTGHDVDFLITSPEATE
DEEQQLLHKVTDFWKQQGLLLYGDILESTFEKFKQPSRKVDALDHFQKA
FLILKLDHGRVHSEKSGQQEGKGWKAIRVDLVMAPYDRRAFALLGWTGS
RQFERDLRRYATHERKMMLDNHALYDRTKRVFLEAESEEEIFAHLGLDY
IEPWERNA*

### Production of Template Independent DNA Polymerase-SSB Complex

This disclosure provides supporting methods for production of processive template independent DNA Polymerase variants, macromolecules or complexes. In an exemplary embodiment, the SSB is mEcSSB and the template independent DNA polymerase is mTdT.

In an exemplary attachment and purification procedure, the method of attachment involves covalent crosslinking of the template independent DNA polymerase and the SSB protein. In an exemplary production process, both proteins contain a unique purification tag, for example, His-tag and GST-tag respectively, and the proteins are purified by affinity purification prior to crosslinking.

In an exemplary process, the crosslinking reaction is prepared by mixing the purified SSB and template independent DNA polymerase in an equimolar ratio in the presence of excess crosslinking reagent. In an exemplary embodiment, the crosslinking agent is a bifunctional crosslinker, for example, a homobifunctional crosslinker, or a heterobifunctional crosslinker. Preferably, the bifunctional crosslinker is a homobifunctional maleimide crosslinker, for example, bismaleimidoethane, 1,4-bismaleimidobutane, bismaleimidohexane, 1,8-bismaleimido-diethyleneglycol, 1,11-bismaleimido-triethyleneglycol, and the like. Methods for covalent chemical conjugation to proteins are known to those of skill in the art (See, e.g., Hermanson, G. T. (2013). Bioconjugate Techniques. Academic Press, hereby incorporated by reference in its entirety). In an exemplary maleimide crosslinking reaction, a 2-3 fold molar excess of crosslinker to protein target is sufficient, although other molar ratios may be used. In other aspects of maleimide crosslinking, the concentration of protein in the reaction is preferably in the range of 0.1 mM, although other conditions are permissible. In other embodiments of maleimide crosslinking, the reaction proceeds for 2 hours at 4° C. in a sulfhydryl-free buffering media, such as phosphate buffered saline at pH 6.5-7.5.

After the crosslinking reaction is completed, the crosslinker is quenched. In an exemplary process of maleimide crosslinking, the quenching reagent can be cysteine, DTT, or other thiol-containing reducing agent, added at 100-500 fold molar excess at final concentration, for example, 10 mM to 50 mM. In other embodiments, the maleimide reaction quenching may proceed for 15 minutes at room temperature, however alternative conditions may be used.

After a crosslinking reaction is quenched, the crosslinked SSB-template independent DNA polymerase product can be purified by several methods. In an exemplary purification process, the method of purification occurs in two steps. In the first step of an exemplary purification process, the quenched, crosslinked product is initially affinity purified by the SSB affinity tag, for example, on GST column if the SSB is GST tagged. In the second step of an exemplary purification process, the SSB elution product is purified on an affinity column selective to the template independent DNA polymerase affinity Tag. For example, if the template independent DNA polymerase contains a His-tag, then concentrated GST tagged elution product may be purified on a His-tag selective Ni-NTA column. In further exemplary embodiments of a purification process, the elution of the captured template independent DNA polymerase complex product from the affinity column can be dialyzed, which should be sufficiently pure for use.

In certain embodiments of affinity chromatography, it may be advantageous to remove large affinity tags before or during protein purification. For example, it can be advantageous to remove GST tags from proteins of interest, prior to use. In such cases, a protease recognition sequence may be introduced between the protein and affinity tag. In an exemplary process, a GST-TEV tag is removed from a template independent DNA binding protein, by on column protease cleavage as the first or second step of purification.

Alternatively, other purification processes may be suitable to purify the quenched crosslinked protein complexes products. In an exemplary process, provided that there is a large molecular difference between the sum of the proteins of the complex, and the individual proteins, size exclusion membranes or columns can be used.

### Material and Methods

### Protein Expression

Gene blocks comprising the His-tagged mutant murine terminal nucleotidyl transferase (mTDT) gene sequences were ordered and shipped from Integrated DNA Technologies, Inc.

Sequences were then inserted into the commercially available pET28b bacterial expression plasmid using a Gibson Assembly master mix (New England Biolabs). A reaction containing 0.125 pmol of expression plasmid and 0.375 pmol of mTDT insert sequence was incubated at 50° C. for 2 hours and then placed on ice for subsequent transformation into T7 Express Chemically Competent E. coli (New England Biolabs).

A vial containing 50 μL of chemically competent bacteria was transformed using approximately 50 ng of the fully assembled expression plasmid directly from the Gibson Assembly reaction tube following the protocol outlined by the manufacturer. Briefly, plasmid and chemically competent cells were incubated on ice for 30 minutes without mixing and then heat shocked at 42° C. for 30 seconds. The cells were then placed on ice for another 5 minutes and 950 μL of SOC media was added to the vial before shaking at 200 RPM for 1 hour at 37° C. Transformed cells were selected for using LB-Kanamycin agar plates with an antibiotic concentration of 50 pg/mL.

The plates were incubated overnight at 37° C. and the resulting colonies were picked for Sanger Sequencing in order to verify that the insert sequence for the His-tagged mutant mTDT was correct (Genewiz, Inc). Colonies that contained perfect copies of the insert sequences were used to inoculate 2 mL of LB-Kanamycin media and were incubated at 37° C. overnight shaking at 200 RPM. The resulting cell suspension was diluted 1:400 in flasks containing 30 mL of fresh LB-Kanamycin media and allowed to reach OD 0.6 (600 nm) while incubating at 37° C. shaking at 200 RPM for approximately 2-3 hours. Expression of the His-tagged mTDT protein was induced by adding Isopropyl β-D-1-thiogalactopyranoside (IPTG) (Sigma) to the cell suspension at a concentration of 1 mM. Flasks were then incubated at 15° C. while shaking at 200 RPM overnight.

### Protein Purification

The His-tagged mutant mTDT produced by the induced bacterial cell suspension was purified with immobilized metal affinity chromatography using a Clontech HisTalon Gravity Column Kit as per manufacturer's instructions (Takara Bio USA). Briefly, cell suspensions were pelleted after spinning samples at 3000×G for 30 minutes at 4° C. Pellets were then lysed using buffers provided by the HisTalon Kit and incubated with beads containing the metal affinity resin at 4° C. in the presence of protease inhibitor. His-tagged mTDT was eluted from beads and elution fractions were concentrated using 30K MWCO centrifuge filter (EMD Millipore) spun at 5000×G for 30 minutes. The elution buffer was exchanged with 1× Terminal Transferase storage buffer as per recommendation from NEB (50 mM KPO₄, 100 mM NaCl, 1.43 β-ME, 50% Glycerol, 0.1% Triton X-100, pH 7.3 at 25° C.). The concentration of the resultant purified His-tagged mTDT protein was determined colorimetrically with a Reducing Agent Compatible micro BCA kit (Thermo Scientific) and the efficiency of the purification was accessed with denaturing Tris-Glycine gel electrophoresis visualized by Coomassie Orange Flour stain (Thermo Scientific).

### Enzymatic Activity Assay

The activity of the purified His-tagged mTDT protein was accessed by its ability to produce single stranded DNA (ssDNA) and extend an initiator 20-mer oligonucleotide in the presence of a single nucleotide or mixture of different nucleotides. Extension reactions were supplemented with 1× Terminal Transferase Buffer (50 mM Potassium Acetate, 20 mM Tris-Acetate, 10 mM Magnesium Acetate, pH 7.9 at 25° C.), 0.25 mM CoCl₂, and 1× GelStar ssDNA SYBR Dye (Lonza), 5 pmol of Cy3-labeled initiator oligonucleotide and 5000 pmol of dNTPs. 100 ng of purified His-Tagged mTDT was then added to catalyze the extension reaction.

Extension reactions were incubated at 37° C. and monitored on a Biorad CFX96 Touch Real Time PCR Detection System scanning the relative fluorescence units in the SYBR Green channel every 1 minutes. Extension reactions reached completion after approximately 60 to 90 minutes after all nucleotides are depleted. ssDNA extension products were evaluated using denaturing TBE-Urea gel electrophoresis and further visualized on Typhoon FLA Biomolecular Imager (GE Heathcare) in the Cy3 channel.

### Example II

Aspects of the present disclosure are directed to mutant TdT which exhibits enhanced or increased or improved or raised processivity as described herein. According to one aspect a mutant human TdT is described where alanine is present at position 454 instead of arginine. Arginine is naturally present at position 454 in human TdT. Accordingly, mutant R454A hTdT is provided.

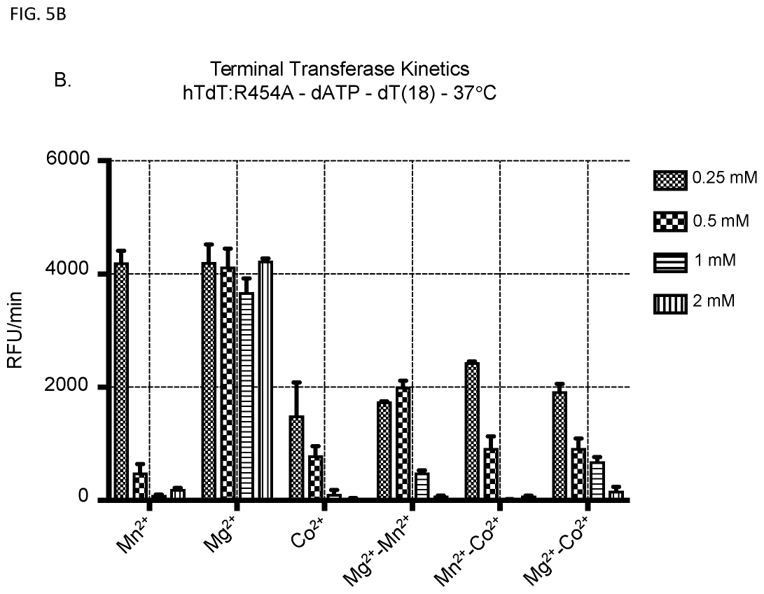

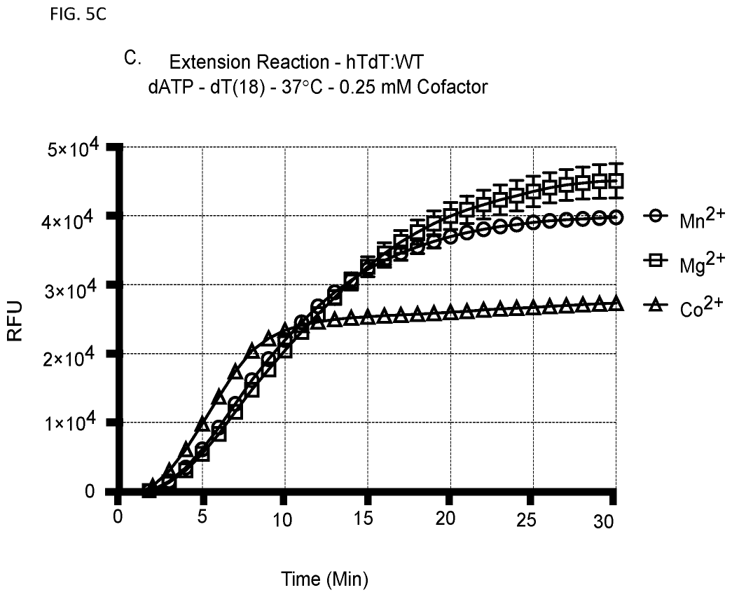

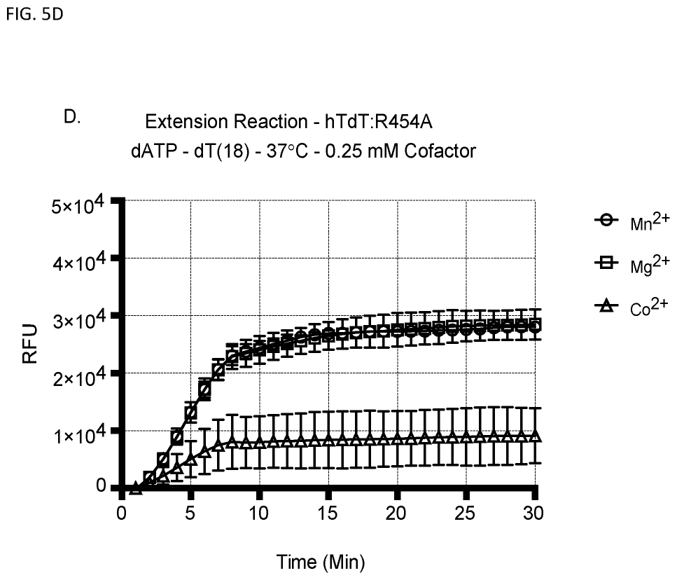

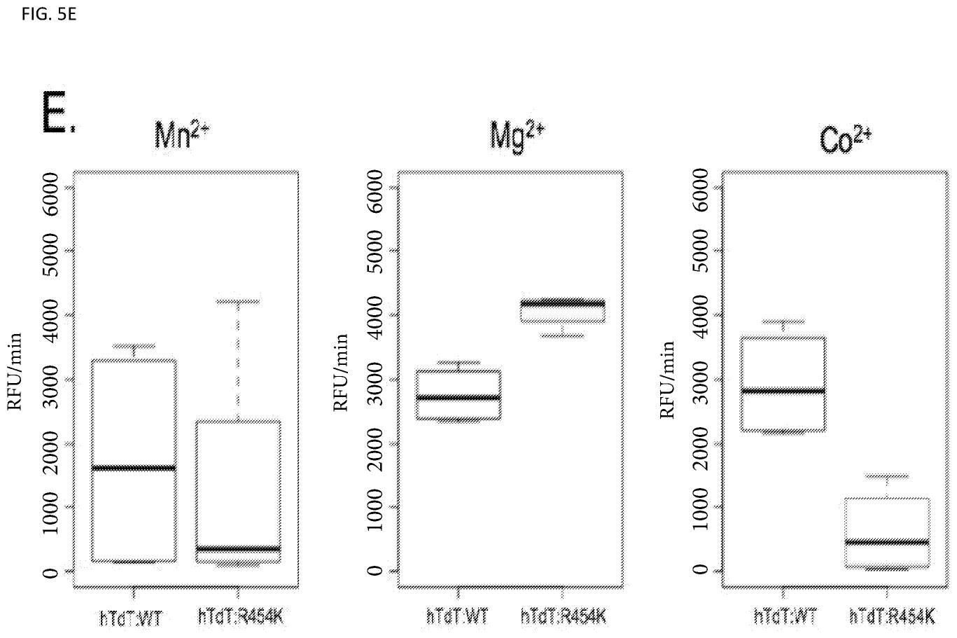

Site directed mutagenesis from arginine to alanine at position 454 (R454A) was performed on a wild-type (WT) human TdT to determine if a mutation to a highly conserved amino acid residue within the catalytic pocket would produce a functional enzyme. According to one aspect, one or more mutations are made within the catalytic pocket and the resulting mutant is a functional TdT enzyme. According to one aspect, the one or more mutations within the catalytic pocket enhance, increase or improve or raise the processivity of wild-type Terminal deoxynucleotidyl Transferases. According to one aspect, the one or more mutations includes R454A. After His-tag purification, WT and R454A hTdT were screened for enzymatic activity with various divalent cations at different concentrations in order to determine the optimal reaction conditions. Combinations of divalent cations such as Mn²⁺ and Co²′, were at a 1:1 ratio for the indicated concentration (0.25 mM-2 mM). A poly-dT (18) initiator oligonucleotide was used at 10 pmol and dATP was supplemented into the reaction at 100 uM. As shown in FIG. 5A, the overall rates of enzyme activity are plotted for the WT hTdT. As shown in FIG. 5B, the overall rates of enzyme activity are plotted for hTdT R454A. These rates were derived from the RFU for ssDNA production measured in real time over 30 minutes as shown in FIG. 5C for WT hTdT and FIG. 5D hTdT R454A (Note: only Mn²⁺, Mg²⁺, and Co²⁺ are shown for simplicity). As shown in FIG. 5E, the average rate of ssDNA production for all divalent cation concentrations were compared for Mn²⁺, Mg²⁺, and Co²⁺ between the WT hTdT and hTDT454A.

### Example III

### Material and Methods

### General Protein Expression

The primary sequences of wild-type or mutant enzymes of interest such as mTdT 388 and WT hTdT were codon optimized for an E. coli expression system using a custom optimization algorithm and ordered as gBlocks® (Integrated DNA Technologies). Sequences were then inserted into the commercially available pET-28-c-(+) His-tag expression vector (EMD Millipore) using a Gibson Assembly master mix (New England Biolabs). A reaction containing 0.125 pmol of expression plasmid and 0.375 pmol of the gBlock® insert sequence was incubated at 50° C. for 2 hours and then transformed into T7 Express Chemically Competent E. coli (New England Biolabs). A vial containing 50 μL of chemically competent bacteria was transformed using approximately 50 ng of the fully assembled expression plasmid directly from the Gibson Assembly reaction tube following the protocol outlined by the manufacturer. Briefly, plasmid and chemically competent cells were incubated on ice for 30 minutes without mixing and then heat shocked at 42° C. for 30 seconds. The cells were then placed on ice for another 5 minutes and 950 μL of SOC media was added to the vial before shaking at 200 RPM for 1 hour at 37° C. Transformed cells were selected for using LB-Kanamycin agar plates with an antibiotic concentration of 50 pg/mL.

The plates were incubated overnight at 37° C. and the resulting colonies were picked for Sanger Sequencing in order to verify that the insert sequence for the His-tag enzyme of interest was correct (Genewiz, Inc). Colonies that contained perfect copies of the insert sequences were used to inoculate 2 mL of LB-Kanamycin media and were incubated at 37° C. overnight shaking at 200 RPM. The resulting cell suspension was diluted 1:400 in flasks containing 30 mL of fresh LB-Kanamycin media and allowed to reach OD 0.6 (600 nm) while incubating at 37° C. shaking at 200 RPM for approximately 2-3 hours. Expression of the His-tag enzyme of interest was induced by adding Isopropyl β-D-1-thiogalactopyranoside (IPTG) (Sigma) to the cell suspension at a concentration of 1 mM. Flasks were then incubated at 15° C. while shaking at 200 RPM overnight.

### General Protein Purification

The His-tag enzyme of interest produced by the induced bacterial cell suspension was purified with immobilized metal affinity chromatography using a Clontech HisTalon Gravity Column Kit as per manufacturer's instructions (Takara Bio USA). Briefly, cell suspensions were pelleted after spinning samples at 3000×G for 30 minutes at 4° C. Pellets were then lysed using buffers provided by the HisTalon Kit and incubated with beads containing the metal affinity resin at 4° C. in the presence of protease inhibitor. His-tagged enzyme of interest was eluted from beads and elution fractions were concentrated using 30K MWCO centrifuge filter (EMD Millipore) spun at 5000×G for 30 minutes. For Terminal deoxynucleotidyl Transferases, such as mTdT 388 and WT hTdT, the elution buffer was exchanged with 1× Terminal Transferase storage buffer as per recommendation from NEB (50 mM KPO₄, 100 mM NaCl, 1.43 β-ME, 50% Glycerol, 0.1% Triton X-100, pH 7.3 at 25° C.). The concentration of the resultant purified His-tagged enzyme of interest was determined colorimetrically with a Reducing Agent Compatible micro BCA kit (Thermo Scientific) and the efficiency of the purification was accessed with denaturing Tris-Glycine gel electrophoresis visualized by Coomassie Orange Flour stain (Thermo Scientific).

### General Site Directed Mutagenesis

Plasmids carrying the target protein were harvested and purified from a sequence verified liquid bacterial cultures grown overnight in LB-kanamycin media at 37 C using a MiniPrep Kit (Qiagen). Oligonucleotide primers were ordered from IDT and were designed to PCR amplify the protein expression plasmid while simultaneously mutagenizing the plasmid at the predetermined location, yielding linearized DNA. Using the reagents from the Q5 Site-Directed Mutagenesis Kit (NEB), the protein expression plasmid was PCR amplified using the Q5 Hot Start High-Fidelity 2× Master Mix with the following thermocycling conditions: initial denature for 98° C. for 30 seconds, denature at 98° C. for 10 seconds, anneal at 68° C. for 10 seconds, and extend at 72° C. for 120 seconds for 25 cycles before a final extension of 2 minutes at 72° C. 1 uL of the resulting PCR amplification reaction was then treated with the kit's enzyme reaction cocktail to re-circularize the protein expression plasmid while digesting away the unsubstituted plasmid sequences remaining in the reaction mixture. After bacterial transformation and sequence verification, colonies with perfect sequence matches were used to express the site-directed mutant protein.

### Enzymatic Activity Assay (Terminal Transferase Kinetic Assay)

The enzymatic activity of purified mTdT 388 and WT hTdT was accessed by their ability to produce single stranded DNA (ssDNA) and extend an initiator 18-mer oligonucleotide in the presence of a single nucleotide or mixture of different nucleotides. Extension reactions were supplemented with 1× Terminal Transferase Buffer (50 mM Potassium Acetate, 20 mM Tris-Acetate, 10 mM Magnesium Acetate, pH 7.9 at 25° C.), 0.25 mM CoCl₂ (or equivalent), and 1× GelStar ssDNA SYBR Dye (Lonza), 10 pmol of Cy3-labeled initiator oligonucleotide and 100 uM of dNTPs. 1 uL of purified mTdT 388 or WT hTdT was then added to catalyze the extension reaction. Extension reactions were incubated at 37° C. and monitored on a Biorad CFX96 Touch Real Time PCR Detection System scanning the relative fluorescence units in the SYBR Green channel every 1 minutes for 30 minutes. The ssDNA extension products from these reactions were evaluated using denaturing 15% TBE-Urea gel electrophoresis and visualized on Typhoon FLA Biomolecular Imager (GE Heathcare) in the Cy3 channel.

1. An enzymatic method of making a polynucleotide comprising
combining a selected nucleotide triphosphate, one or more cations, a template-independent polymerase, and an associated processivity factor in an aqueous reaction medium including a target substrate comprising an initiator sequence and having a 3′ terminal nucleotide attached to a single stranded portion, such that the template-independent polymerase and the associated processivity factor interact with the target substrate under conditions which covalently add one or more of the selected nucleotide triphosphate to the 3′ terminal nucleotide.

2. The method of claim 1 further including
repeatedly introducing a subsequent selected nucleotide triphosphate to the aqueous reaction medium under conditions which enzymatically add one or more of the subsequent selected nucleotide triphosphate to the target substrate until the polynucleotide is formed.

3. The method of claim 1 wherein the processivity factor increases processivity of the template-independent polymerase.

4. The method of claim 1 wherein the processivity factor comprises one or more binding units.

5. The method of claim 1 wherein the processivity factor binds to and translocates across the target substrate.

6. The method of claim 1 wherein the processivity factor binds to and reptates across the target substrate.

7. The method of claim 1 wherein the processivity factor and the template-independent polymerase bind to the target substrate.

8. The method of claim 1 wherein the processivity factor and the template-independent polymerase bind to the target substrate with an affinity greater than the template-independent polymerase alone.

9. The method of claim 1 wherein the processivity factor and the template-independent polymerase comprise a fusion protein.

10. The method of claim 1 wherein the processivity factor is attached to the template-independent polymerase at a location on the template-independent polymerase which facilitates processing of the target substrate by the template-independent polymerase.

11. The method of claim 1 wherein the processivity factor is attached by a covalent or noncovalent bond to the template-independent polymerase at a location on the template-independent polymerase which facilitates processing of the target substrate by the template-independent polymerase.

12. The method of claim 1 wherein the processivity factor is attached to the template-independent polymerase through a linker at a location on the template-independent polymerase which facilitates processing of the target substrate by the template-independent polymerase.

13. The method of claim 1 wherein the processivity factor includes a polypeptide binding domain that binds to the template-independent polymerase.

14. The method of claim 1 wherein the template-independent polymerase includes a polypeptide binding domain that binds to the processivity factor.

15. The method of claim 1 wherein the template-independent polymerase and the processivity factor each include one member of a binding pair wherein the template-independent polymerase and the processivity factor are attached via the binding pair.

16. The method of claim 1 wherein the template-independent polymerase and the processivity factor are crosslinked via a crosslinker.

17. The method of claim 1 wherein the template-independent polymerase and the processivity factor are crosslinked via sulfhydryl crosslinking.

18. The method of claim 1 wherein the template-independent polymerase and the processivity factor are attached via protein conjugation.

19. The method of claim 1 wherein the template-independent polymerase and the processivity factor are immobilized relative to one another in an orientation which facilitates processing of the substrate by the template-independent polymerase.

20. The method of claim 1 wherein the template-independent polymerase and the processivity factor are immobilized relative to one another on a substrate in an orientation which facilitates processing of the substrate by the template-independent polymerase.

21. The method of claim 1 wherein the template-independent polymerase and the processivity factor are co-localized on a substrate in an orientation which facilitates processing of the substrate by the template-independent polymerase.

22. The method of claim 1 wherein the template-independent polymerase is a template-independent DNA or RNA polymerase.

23. The method of claim 1 wherein the template-independent polymerase is a template-independent DNA polymerase.

24. The method of claim 1 wherein the template-independent polymerase is a terminal deoxynucleotidyl transferase (TdT).

25. The method of claim 1 wherein the template-independent polymerase is a TdT of the polX family of DNA polymerases.

26. The method of claim 24 wherein the TdT a mammalian TdT.

27. The method of claim 24 wherein the TdT is a member of the archaeo-eukaryotic primase (AEP) superfamily.

28. The method of claim 24 wherein the TdT is a PolpTN2 or a C-terminal truncated PolpTN2, a PriS, a nonhomologous end joining archaeo-eukaryotic primase, a mammalian Polθ, or a eukaryotic PrimPol.

29. The method of claim 1 wherein the template-independent polymerase is a mutant where one or more cysteine residues are replaced by one or more non-cysteine residues.

30. The method of claim 1 wherein the template-independent polymerase is a mutant where all naturally occurring cysteine residues are replaced by one or more non-cysteine residues.

31. The method of claim 1 wherein the template-independent polymerase is a mutant where all naturally occurring cysteine residues are replaced by one or more non-cysteine residues and a surface accessible cysteine residue is provided.

32. The method of claim 1 wherein the template-independent polymerase is a mutant where one or more non-cysteine residues are replaced by one or more cysteine residues.

33. The method of claim 1 wherein the template-independent polymerase is a mutant having one or more one surface accessible cysteine residues.

34. The method of claim 1 wherein the template-independent polymerase is a mutant having at most one surface accessible cysteine residue.

35. The method of claim 1 wherein the template-independent polymerase and the processivity factor each include a mutant surface accessible cysteine residue which connect the template-independent polymerase to the processivity factor.

36. The method of claim 1 wherein the processivity factor comprises a prokaryotic or eukaryotic single stranded DNA binding protein.

37. The method of claim 1 wherein the processivity factor comprises a prokaryotic single stranded DNA binding protein.

38. The method of claim 1 wherein the processivity factor comprises an E. coli single stranded DNA binding protein.

39. A mutant template-independent polymerase having one or more mutations from a cysteine residue to a non-cysteine residue.

40. A mutant template-independent polymerase having one or more mutations from a non-cysteine residue to a cysteine residue.

41. A mutant template-independent polymerase having one or more mutations from a non-cysteine residue to a surface accessible cysteine residue.

42. A mutant template-independent polymerase having a mutation from a non-cysteine residue to a surface accessible cysteine residue.

43. A mutant template-independent polymerase having a mutation from a non-cysteine residue to at most one surface accessible cysteine residue.

44. A macromolecule comprising a template-independent polymerase having a processivity factor attached thereto.

45. The macromolecule of claim 44 wherein the template-independent polymerase is a template-independent DNA or RNA polymerase.

46. The macromolecule of claim 44 wherein the template-independent polymerase is a template-independent DNA polymerase.

47. The macromolecule of claim 44 wherein the template-independent polymerase is a terminal deoxynucleotidyl transferase (TdT).

48. The macromolecule of claim 46 wherein the template-independent polymerase is a TdT of the polX family of DNA polymerases.

49. The macromolecule of claim 46 wherein the TdT a mammalian TdT.

50. The macromolecule of claim 46 wherein the TdT is a member of the archaeo-eukaryotic primase (AEP) superfamily.

51. The macromolecule of claim 46 wherein the TdT is a PolpTN2 or a C-terminal truncated PolpTN2, a PriS, a nonhomologous end joining archaeo-eukaryotic primase, a mammalian Polθ, or a eukaryotic PrimPol.

52. The macromolecule of claim 44 wherein the template-independent polymerase is a mutant where one or more cysteine residues is replaced by a non-cysteine residue.

53. The macromolecule of claim 44 wherein the template-independent polymerase is a mutant where one or more non-cysteine residues is replaced by a cysteine residue.

54. The macromolecule of claim 44 wherein the template-independent polymerase is a mutant having one or more one surface accessible cysteine residues.

55. The macromolecule of claim 44 wherein the template-independent polymerase is a mutant having at most one surface accessible cysteine residue.

56. The macromolecule of claim 44 wherein the template-independent polymerase is attached to the processivity factor by a mutant surface accessible cysteine residue.

57. The macromolecule of claim 44 wherein the processivity factor comprises a prokaryotic or eukaryotic single stranded DNA binding protein.

58. The macromolecule of claim 44 wherein the processivity factor comprises a prokaryotic single stranded DNA binding protein.

59. The macromolecule of claim 44 wherein the processivity factor comprises an E. coli single stranded DNA binding protein.

60. The macromolecule of claim 44 wherein the template-independent polymerase and the processivity factor comprise a fusion protein.

61. The macromolecule of claim 60 wherein fusion protein comprises SEQ ID NO. 1 and SEQ ID NO. 2.

62. A system for making a polynucleotide comprising
a selected nucleotide triphosphate, one or more cations, a template-independent polymerase, and an associated processivity factor in an aqueous reaction medium including a target substrate comprising an initiator sequence and having a 3′ terminal nucleotide attached to a single stranded portion.
